SEQUENCE LISTING (101 sequences)

SEQ ID NO: 1 - DNA, 17 bp, Artificial Sequence
  Description of Artificial Sequence: Synthetic primer
  modified_base (1)..(6): a, c, t, g, unknown or other
nnnnnngaattgggtta

SEQ ID NO: 2 - DNA, 17 bp, Artificial Sequence
  Description of Artificial Sequence: Synthetic primer
  modified_base (1)..(6): a, c, t, g, unknown or other
nnnnnncagcactgctc

SEQ ID NO: 3 - DNA, 17 bp, Artificial Sequence
  Description of Artificial Sequence: Synthetic primer
  modified_base (1)..(6): a, c, t, g, unknown or other
nnnnnncctgagttgag

SEQ ID NO: 4 - DNA, 17 bp, Artificial Sequence
  Description of Artificial Sequence: Synthetic primer
  modified_base (1)..(6): a, c, t, g, unknown or other
nnnnnnagttgagtcag

SEQ ID NO: 5 - DNA, 17 bp, Artificial Sequence
  Description of Artificial Sequence: Synthetic primer
  modified_base (1)..(6): a, c, t, g, unknown or other
nnnnnnagtcagcactg

SEQ ID NO: 6 - DNA, 17 bp, Artificial Sequence
  Description of Artificial Sequence: Synthetic primer
  modified_base (1)..(6): a, c, t, g, unknown or other
nnnnnngagtcagcact

SEQ ID NO: 7 - DNA, 17 bp, Artificial Sequence
  Description of Artificial Sequence: Synthetic primer
  modified_base (1)..(6): a, c, t, g, unknown or other
nnnnnngttgagtcagc

SEQ ID NO: 8 - DNA, 17 bp, Artificial Sequence
  Description of Artificial Sequence: Synthetic primer
  modified_base (1)..(6): a, c, t, g, unknown or other
nnnnnnggcctgagttg

SEQ ID NO: 9 - DNA, 17 bp, Artificial Sequence
  Description of Artificial Sequence: Synthetic primer
  modified_base (1)..(6): a, c, t, g, unknown or other
nnnnnngtcagcactgc

SEQ ID NO: 10 - DNA, 23 bp, Artificial Sequence
  Description of Artificial Sequence: Synthetic primer
tgcaattattccccatgaacgag

SEQ ID NO: 11 - DNA, 23 bp, Artificial Sequence
  Description of Artificial Sequence: Synthetic primer
ctagatagtcaagttcgaccgtc

SEQ ID NO: 12 - DNA, 20 bp, Artificial Sequence
  Description of Artificial Sequence: Synthetic primer
ttcggcttcgtggttaaact

SEQ ID NO: 13 - DNA, 20 bp, Artificial Sequence
  Description of Artificial Sequence: Synthetic primer
ctcttcctggctccttctca

SEQ ID NO: 14 - DNA, 20 bp, Artificial Sequence
  Description of Artificial Sequence: Synthetic primer
ggatgcctcctttgccggag

SEQ ID NO: 15 - DNA, 20 bp, Artificial Sequence
  Description of Artificial Sequence: Synthetic primer
agccattgaactcacttcgc

SEQ ID NO: 16 - DNA, 21 bp, Artificial Sequence
  Description of Artificial Sequence: Synthetic primer
agtcaagcctcttctcgttcc

SEQ ID NO: 17 - DNA, 21 bp, Artificial Sequence
  Description of Artificial Sequence: Synthetic primer
gcaaagcaagagcagcatcac

SEQ ID NO: 18 - DNA, 26 bp, Artificial Sequence
  Description of Artificial Sequence: Synthetic primer
ggtgttaattggaacgccttgtcctc

SEQ ID NO: 19 - DNA, 23 bp, Artificial Sequence
  Description of Artificial Sequence: Synthetic primer
tcttggttcaccgctctcactca

SEQ ID NO: 20 - DNA, 68 bp, Artificial Sequence
  Description of Artificial Sequence: Synthetic primer
  modified_base (49)..(51): a, c, t, g, unknown or other
taatacgactcactatagggccgatatccgacggtagtgtcacgtcgtnnnatcatccaaatctgcag

SEQ ID NO: 21 - DNA, 68 bp, Artificial Sequence
  Description of Artificial Sequence: Synthetic primer
  modified_base (49)..(51): a, c, t, g, unknown or other
taatacgactcactatagggccgatatccgacggtagtgtcaattgatnnnatcatccaaatctgcag

SEQ ID NO: 22 - DNA, 68 bp, Artificial Sequence
  Description of Artificial Sequence: Synthetic primer
  modified_base (49)..(51): a, c, t, g, unknown or other
taatacgactcactatagggccgatatccgacggtagtgtatattgtannnatcatccaaatctgcag

SEQ ID NO: 23 - DNA, 68 bp, Artificial Sequence
  Description of Artificial Sequence: Synthetic primer
  modified_base (49)..(51): a, c, t, g, unknown or other
taatacgactcactatagggccgatatccgacggtagtgtatagcacgnnnatcatccaaatctgcag

SEQ ID NO: 24 - DNA, 68 bp, Artificial Sequence
  Description of Artificial Sequence: Synthetic primer
  modified_base (49)..(51): a, c, t, g, unknown or other
taatacgactcactatagggccgatatccgacggtagtgtacacatgtnnnatcatccaaatctgcag

SEQ ID NO: 25 - DNA, 68 bp, Artificial Sequence
  Description of Artificial Sequence: Synthetic primer
  modified_base (49)..(51): a, c, t, g, unknown or other
taatacgactcactatagggccgatatccgacggtagtgtatgtaatgnnnatcatccaaatctgcag

SEQ ID NO: 26 - DNA, 68 bp, Artificial Sequence
  Description of Artificial Sequence: Synthetic primer
  modified_base (49)..(51): a, c, t, g, unknown or other
taatacgactcactatagggccgatatccgacggtagtgtagtatctgnnnatcatccaaatctgcag

SEQ ID NO: 27 - DNA, 68 bp, Artificial Sequence
  Description of Artificial Sequence: Synthetic primer
  modified_base (49)..(51): a, c, t, g, unknown or other
taatacgactcactatagggccgatatccgacggtagtgtatgcttgannnatcatccaaatctgcag

SEQ ID NO: 28 - DNA, 68 bp, Artificial Sequence
  Description of Artificial Sequence: Synthetic primer
  modified_base (49)..(51): a, c, t, g, unknown or other
taatacgactcactatagggccgatatccgacggtagtgtaactgtatnnnatcatccaaatctgcag

SEQ ID NO: 29 - DNA, 68 bp, Artificial Sequence
  Description of Artificial Sequence: Synthetic primer
  modified_base (49)..(51): a, c, t, g, unknown or other
taatacgactcactatagggccgatatccgacggtagtgtcaggcattnnnatcatccaaatctgcag

SEQ ID NO: 30 - DNA, 68 bp, Artificial Sequence
  Description of Artificial Sequence: Synthetic primer
  modified_base (49)..(51): a, c, t, g, unknown or other
taatacgactcactatagggccgatatccgacggtagtgtaaggcgatnnnatcatccaaatctgcag

SEQ ID NO: 31 - DNA, 68 bp, Artificial Sequence
  Description of Artificial Sequence: Synthetic primer
  modified_base (49)..(51): a, c, t, g, unknown or other
taatacgactcactatagggccgatatccgacggtagtgtgcgtcgaannnatcatccaaatctgcag

SEQ ID NO: 32 - DNA, 67 bp, Artificial Sequence
  Description of Artificial Sequence: Synthetic primer
  modified_base (48)..(50): a, c, t, g, unknown or other
aatacgactcactatagggccgatatccgacggtagtgtgaacgacannnatcatccaaatctgcag

SEQ ID NO: 33 - DNA, 68 bp, Artificial Sequence
  Description of Artificial Sequence: Synthetic primer
  modified_base (49)..(51): a, c, t, g, unknown or other
taatacgactcactatagggccgatatccgacggtagtgtggcaagcannnatcatccaaatctgcag

SEQ ID NO: 34 - DNA, 68 bp, Artificial Sequence
  Description of Artificial Sequence: Synthetic primer
  modified_base (49)..(51): a, c, t, g, unknown or other
taatacgactcactatagggccgatatccgacggtagtgtgtaaccgannnatcatccaaatctgcag

SEQ ID NO: 35 - DNA, 68 bp, Artificial Sequence
  Description of Artificial Sequence: Synthetic primer
  modified_base (49)..(51): a, c, t, g, unknown or other
taatacgactcactatagggccgatatccgacggtagtgtgctatggannnatcatccaaatctgcag

SEQ ID NO: 36 - DNA, 68 bp, Artificial Sequence
  Description of Artificial Sequence: Synthetic primer
  modified_base (49)..(51): a, c, t, g, unknown or other
taatacgactcactatagggccgatatccgacggtagtgtgacacttannnatcatccaaatctgcag

SEQ ID NO: 37 - DNA, 68 bp, Artificial Sequence
  Description of Artificial Sequence: Synthetic primer
  modified_base (49)..(51): a, c, t, g, unknown or other
taatacgactcactatagggccgatatccgacggtagtgtggttggacnnnatcatccaaatctgcag

SEQ ID NO: 38 - DNA, 68 bp, Artificial Sequence
  Description of Artificial Sequence: Synthetic primer
  modified_base (49)..(51): a, c, t, g, unknown or other
taatacgactcactatagggccgatatccgacggtagtgttcagattcnnnatcatccaaatctgcag

SEQ ID NO: 39 - DNA, 68 bp, Artificial Sequence
  Description of Artificial Sequence: Synthetic primer
  modified_base (49)..(51): a, c, t, g, unknown or other
taatacgactcactatagggccgatatccgacggtagtgttatgccagnnnatcatccaaatctgcag

SEQ ID NO: 40 - DNA, 68 bp, Artificial Sequence
  Description of Artificial Sequence: Synthetic primer
  modified_base (49)..(51): a, c, t, g, unknown or other
taatacgactcactatagggccgatatccgacggtagtgttggctcagnnnatcatccaaatctgcag

SEQ ID NO: 41 - DNA, 68 bp, Artificial Sequence
  Description of Artificial Sequence: Synthetic primer
  modified_base (49)..(51): a, c, t, g, unknown or other
taatacgactcactatagggccgatatccgacggtagtgttcattgagnnnatcatccaaatctgcag

SEQ ID NO: 42 - DNA, 68 bp, Artificial Sequence
  Description of Artificial Sequence: Synthetic primer
  modified_base (49)..(51): a, c, t, g, unknown or other
taatacgactcactatagggccgatatccgacggtagtgttgtatgcgnnnatcatccaaatctgcag

SEQ ID NO: 43 - DNA, 68 bp, Artificial Sequence
  Description of Artificial Sequence: Synthetic primer
  modified_base (49)..(51): a, c, t, g, unknown or other
taatacgactcactatagggccgatatccgacggtagtgttccagtcgnnnatcatccaaatctgcag

SEQ ID NO: 44 - DNA, 68 bp, Artificial Sequence
  Description of Artificial Sequence: Synthetic primer
  modified_base (49)..(51): a, c, t, g, unknown or other
taatacgactcactatagggccgatatccgacggtagtgttacttcggnnnatcatccaaatctgcag

SEQ ID NO: 45 - DNA, 68 bp, Artificial Sequence
  Description of Artificial Sequence: Synthetic primer
  modified_base (49)..(51): a, c, t, g, unknown or other
taatacgactcactatagggccgatatccgacggtagtgttgaactggnnnatcatccaaatctgcag

SEQ ID NO: 46 - DNA, 68 bp, Artificial Sequence
  Description of Artificial Sequence: Synthetic primer
  modified_base (49)..(51): a, c, t, g, unknown or other
taatacgactcactatagggccgatatccgacggtagtgtttggtatgnnnatcatccaaatctgcag

SEQ ID NO: 47 - DNA, 68 bp, Artificial Sequence
  Description of Artificial Sequence: Synthetic primer
  modified_base (49)..(51): a, c, t, g, unknown or other
taatacgactcactatagggccgatatccgacggtagtgttaacgctgnnnatcatccaaatctgcag

SEQ ID NO: 48 - DNA, 68 bp, Artificial Sequence
  Description of Artificial Sequence: Synthetic primer
  modified_base (49)..(51): a, c, t, g, unknown or other
taatacgactcactatagggccgatatccgacggtagtgtttccattgnnnatcatccaaatctgcag

SEQ ID NO: 49 - DNA, 68 bp, Artificial Sequence
  Description of Artificial Sequence: Synthetic primer
  modified_base (49)..(51): a, c, t, g, unknown or other
taatacgactcactatagggccgatatccgacggtagtgttgtggttgnnnatcatccaaatctgcag

SEQ ID NO: 50 - DNA, 68 bp, Artificial Sequence
  Description of Artificial Sequence: Synthetic primer
  modified_base (49)..(51): a, c, t, g, unknown or other
taatacgactcactatagggccgatatccgacggtagtgttacaggatnnnatcatccaaatctgcag

SEQ ID NO: 51 - DNA, 68 bp, Artificial Sequence
  Description of Artificial Sequence: Synthetic primer
  modified_base (49)..(51): a, c, t, g, unknown or other
taatacgactcactatagggccgatatccgacggtagtgtttcctgctnnnatcatccaaatctgcag

SEQ ID NO: 52 - DNA, 68 bp, Artificial Sequence
  Description of Artificial Sequence: Synthetic primer
  modified_base (49)..(51): a, c, t, g, unknown or other
taatacgactcactatagggccgatatccgacggtagtgttgcgatctnnnatcatccaaatctgcag

SEQ ID NO: 53 - DNA, 68 bp, Artificial Sequence
  Description of Artificial Sequence: Synthetic primer
  modified_base (49)..(51): a, c, t, g, unknown or other
taatacgactcactatagggccgatatccgacggtagtgttgcatagtnnnatcatccaaatctgcag

SEQ ID NO: 54 - DNA, 68 bp, Artificial Sequence
  Description of Artificial Sequence: Synthetic primer
  modified_base (49)..(51): a, c, t, g, unknown or other
taatacgactcactatagggccgatatccgacggtagtgttgatacgtnnnatcatccaaatctgcag

SEQ ID NO: 55 - DNA, 68 bp, Artificial Sequence
  Description of Artificial Sequence: Synthetic primer
  modified_base (49)..(51): a, c, t, g, unknown or other
taatacgactcactatagggccgatatccgacggtagtgttcgagcgtnnnatcatccaaatctgcag

SEQ ID NO: 56 - DNA, 67 bp, Artificial Sequence
  Description of Artificial Sequence: Synthetic primer
  modified_base (49)..(51): a, c, t, g, unknown or other
taatacgactcactatagggccgatatccgacggtagtgtgcttcacannngacgggcggtgtgtac

SEQ ID NO: 57 - DNA, 67 bp, Artificial Sequence
  Description of Artificial Sequence: Synthetic primer
  modified_base (49)..(51): a, c, t, g, unknown or other
taatacgactcactatagggccgatatccgacggtagtgtcgatgtttnnngacgggcggtgtgtac

SEQ ID NO: 58 - DNA, 67 bp, Artificial Sequence
  Description of Artificial Sequence: Synthetic primer
  modified_base (49)..(51): a, c, t, g, unknown or other
taatacgactcactatagggccgatatccgacggtagtgtttaggcatnnngacgggcggtgtgtac

SEQ ID NO: 59 - DNA, 67 bp, Artificial Sequence
  Description of Artificial Sequence: Synthetic primer
  modified_base (49)..(51): a, c, t, g, unknown or other
taatacgactcactatagggccgatatccgacggtagtgtacagtggtnnngacgggcggtgtgtac

SEQ ID NO: 60 - DNA, 67 bp, Artificial Sequence
  Description of Artificial Sequence: Synthetic primer
  modified_base (49)..(51): a, c, t, g, unknown or other
taatacgactcactatagggccgatatccgacggtagtgtgccaatgtnnngacgggcggtgtgtac

SEQ ID NO: 61 - DNA, 67 bp, Artificial Sequence
  Description of Artificial Sequence: Synthetic primer
  modified_base (49)..(51): a, c, t, g, unknown or other
taatacgactcactatagggccgatatccgacggtagtgtcagatctgnnngacgggcggtgtgtac

SEQ ID NO: 62 - DNA, 67 bp, Artificial Sequence
  Description of Artificial Sequence: Synthetic primer
  modified_base (49)..(51): a, c, t, g, unknown or other
taatacgactcactatagggccgatatccgacggtagtgtacttgatgnnngacgggcggtgtgtac

SEQ ID NO: 63 - DNA, 67 bp, Artificial Sequence
  Description of Artificial Sequence: Synthetic primer
  modified_base (49)..(51): a, c, t, g, unknown or other
taatacgactcactatagggccgatatccgacggtagtgttagcttgtnnngacgggcggtgtgtac

SEQ ID NO: 64 - DNA, 67 bp, Artificial Sequence
  Description of Artificial Sequence: Synthetic primer
  modified_base (49)..(51): a, c, t, g, unknown or other
taatacgactcactatagggccgatatccgacggtagtgttggttgttnnngacgggcggtgtgtac

SEQ ID NO: 65 - DNA, 67 bp, Artificial Sequence
  Description of Artificial Sequence: Synthetic primer
  modified_base (49)..(51): a, c, t, g, unknown or other
taatacgactcactatagggccgatatccgacggtagtgttgtaccttnnngacgggcggtgtgtac

SEQ ID NO: 66 - DNA, 67 bp, Artificial Sequence
  Description of Artificial Sequence: Synthetic primer
  modified_base (49)..(51): a, c, t, g, unknown or other
taatacgactcactatagggccgatatccgacggtagtgttctgctgtnnngacgggcggtgtgtac

SEQ ID NO: 67 - DNA, 67 bp, Artificial Sequence
  Description of Artificial Sequence: Synthetic primer
  modified_base (49)..(51): a, c, t, g, unknown or other
taatacgactcactatagggccgatatccgacggtagtgtttggaggtnnngacgggcggtgtgtac

SEQ ID NO: 68 - DNA, 67 bp, Artificial Sequence
  Description of Artificial Sequence: Synthetic primer
  modified_base (49)..(51): a, c, t, g, unknown or other
taatacgactcactatagggccgatatccgacggtagtgttcgagcgtnnngacgggcggtgtgtac

SEQ ID NO: 69 - DNA, 67 bp, Artificial Sequence
  Description of Artificial Sequence: Synthetic primer
  modified_base (49)..(51): a, c, t, g, unknown or other
taatacgactcactatagggccgatatccgacggtagtgttgatacgtnnngacgggcggtgtgtac

SEQ ID NO: 70 - DNA, 67 bp, Artificial Sequence
  Description of Artificial Sequence: Synthetic primer
  modified_base (49)..(51): a, c, t, g, unknown or other
taatacgactcactatagggccgatatccgacggtagtgttgcatagtnnngacgggcggtgtgtac

SEQ ID NO: 71 - DNA, 67 bp, Artificial Sequence
  Description of Artificial Sequence: Synthetic primer
  modified_base (49)..(51): a, c, t, g, unknown or other
taatacgactcactatagggccgatatccgacggtagtgttgcgatctnnngacgggcggtgtgtac

SEQ ID NO: 72 - DNA, 67 bp, Artificial Sequence
  Description of Artificial Sequence: Synthetic primer
  modified_base (49)..(51): a, c, t, g, unknown or other
taatacgactcactatagggccgatatccgacggtagtgtttcctgctnnngacgggcggtgtgtac

SEQ ID NO: 73 - DNA, 67 bp, Artificial Sequence
  Description of Artificial Sequence: Synthetic primer
  modified_base (49)..(51): a, c, t, g, unknown or other
taatacgactcactatagggccgatatccgacggtagtgttacaggatnnngacgggcggtgtgtac

SEQ ID NO: 74 - DNA, 67 bp, Artificial Sequence
  Description of Artificial Sequence: Synthetic primer
  modified_base (49)..(51): a, c, t, g, unknown or other
taatacgactcactatagggccgatatccgacggtagtgttgtggttgnnngacgggcggtgtgtac

SEQ ID NO: 75 - DNA, 67 bp, Artificial Sequence
  Description of Artificial Sequence: Synthetic primer
  modified_base (49)..(51): a, c, t, g, unknown or other
taatacgactcactatagggccgatatccgacggtagtgtttccattgnnngacgggcggtgtgtac

SEQ ID NO: 76 - DNA, 67 bp, Artificial Sequence
  Description of Artificial Sequence: Synthetic primer
  modified_base (49)..(51): a, c, t, g, unknown or other
taatacgactcactatagggccgatatccgacggtagtgttaacgctgnnngacgggcggtgtgtac

SEQ ID NO: 77 - DNA, 67 bp, Artificial Sequence
  Description of Artificial Sequence: Synthetic primer
  modified_base (49)..(51): a, c, t, g, unknown or other
taatacgactcactatagggccgatatccgacggtagtgtttggtatgnnngacgggcggtgtgtac

SEQ ID NO: 78 - DNA, 67 bp, Artificial Sequence
  Description of Artificial Sequence: Synthetic primer
  modified_base (49)..(51): a, c, t, g, unknown or other
taatacgactcactatagggccgatatccgacggtagtgttgaactggnnngacgggcggtgtgtac

SEQ ID NO: 79 - DNA, 67 bp, Artificial Sequence
  Description of Artificial Sequence: Synthetic primer
  modified_base (49)..(51): a, c, t, g, unknown or other
taatacgactcactatagggccgatatccgacggtagtgttacttcggnnngacgggcggtgtgtac

SEQ ID NO: 80 - DNA, 67 bp, Artificial Sequence
  Description of Artificial Sequence: Synthetic primer
  modified_base (49)..(51): a, c, t, g, unknown or other
taatacgactcactatagggccgatatccgacggtagtgttccagtcgnnngacgggcggtgtgtac

SEQ ID NO: 81 - DNA, 67 bp, Artificial Sequence
  Description of Artificial Sequence: Synthetic primer
  modified_base (49)..(51): a, c, t, g, unknown or other
taatacgactcactatagggccgatatccgacggtagtgttgtatgcgnnngacgggcggtgtgtac

SEQ ID NO: 82 - DNA, 67 bp, Artificial Sequence
  Description of Artificial Sequence: Synthetic primer
  modified_base (49)..(51): a, c, t, g, unknown or other
taatacgactcactatagggccgatatccgacggtagtgttcattgagnnngacgggcggtgtgtac

SEQ ID NO: 83 - DNA, 67 bp, Artificial Sequence
  Description of Artificial Sequence: Synthetic primer
  modified_base (49)..(51): a, c, t, g, unknown or other
taatacgactcactatagggccgatatccgacggtagtgttggctcagnnngacgggcggtgtgtac

SEQ ID NO: 84 - DNA, 67 bp, Artificial Sequence
  Description of Artificial Sequence: Synthetic primer
  modified_base (49)..(51): a, c, t, g, unknown or other
taatacgactcactatagggccgatatccgacggtagtgttatgccagnnngacgggcggtgtgtac

SEQ ID NO: 85 - DNA, 67 bp, Artificial Sequence
  Description of Artificial Sequence: Synthetic primer
  modified_base (49)..(51): a, c, t, g, unknown or other
taatacgactcactatagggccgatatccgacggtagtgttcagattcnnngacgggcggtgtgtac

SEQ ID NO: 86 - DNA, 67 bp, Artificial Sequence
  Description of Artificial Sequence: Synthetic primer
  modified_base (49)..(51): a, c, t, g, unknown or other
taatacgactcactatagggccgatatccgacggtagtgtggttggacnnngacgggcggtgtgtac

SEQ ID NO: 87 - DNA, 67 bp, Artificial Sequence
  Description of Artificial Sequence: Synthetic primer
  modified_base (49)..(51): a, c, t, g, unknown or other
taatacgactcactatagggccgatatccgacggtagtgtgacacttannngacgggcggtgtgtac

SEQ ID NO: 88 - DNA, 67 bp, Artificial Sequence
  Description of Artificial Sequence: Synthetic primer
  modified_base (49)..(51): a, c, t, g, unknown or other
taatacgactcactatagggccgatatccgacggtagtgtgctatggannngacgggcggtgtgtac

SEQ ID NO: 89 - DNA, 67 bp, Artificial Sequence
  Description of Artificial Sequence: Synthetic primer
  modified_base (49)..(51): a, c, t, g, unknown or other
taatacgactcactatagggccgatatccgacggtagtgtgtaaccgannngacgggcggtgtgtac

SEQ ID NO: 90 - DNA, 67 bp, Artificial Sequence
  Description of Artificial Sequence: Synthetic primer
  modified_base (49)..(51): a, c, t, g, unknown or other
taatacgactcactatagggccgatatccgacggtagtgtggcaagcannngacgggcggtgtgtac

SEQ ID NO: 91 - DNA, 67 bp, Artificial Sequence
  Description of Artificial Sequence: Synthetic primer
  modified_base (49)..(51): a, c, t, g, unknown or other
taatacgactcactatagggccgatatccgacggtagtgtgaacgacannngacgggcggtgtgtac

SEQ ID NO: 92 - DNA, 26 bp, Artificial Sequence
  Description of Artificial Sequence: Synthetic primer
gatttgtctggttaattccgataacg

SEQ ID NO: 93 - DNA, 20 bp, Artificial Sequence
  Description of Artificial Sequence: Synthetic primer
cgtggtccagaacaaaccca

SEQ ID NO: 94 - DNA, 23 bp, Artificial Sequence
  Description of Artificial Sequence: Synthetic primer
caataacaggtctgtgatgccct

SEQ ID NO: 95 - DNA, 23 bp, Artificial Sequence
  Description of Artificial Sequence: Synthetic primer
tgcaattattccccatgaacgag

SEQ ID NO: 96 - skipped

SEQ ID NO: 97 - DNA, 23 bp, Artificial Sequence
  Description of Artificial Sequence: Synthetic primer
aggtgccatcaaattggatgaca

SEQ ID NO: 98 - DNA, 24 bp, Artificial Sequence
  Description of Artificial Sequence: Synthetic primer
ctgaataagcatattgacgcatac

SEQ ID NO: 99 - DNA, 20 bp, Artificial Sequence
  Description of Artificial Sequence: Synthetic primer
ccgatatccgacggtagtgt

SEQ ID NO: 100 - DNA, 22 bp, Artificial Sequence
  Description of Artificial Sequence: Synthetic primer
gtaaaacgacggccagtgaatt

SEQ ID NO: 101 - DNA, 21 bp, Artificial Sequence
  Description of Artificial Sequence: Synthetic primer
caggaaacagctatgaccatg

(19) United States
(12) Patent Application Publication
(10) Pub. No.: US 2023/0167512 A1
(43) Pub. Date: Jun. 1, 2023

(54) RNA VIRUS DIAGNOSTIC ASSAY

(71) Applicant: Brown University, Providence, RI (US)

(72) Inventor: William FAIRBROTHER, Barrington, RI (US)

(21) Appl. No.: 17/995,785

(22) PCT Filed: Apr. 12, 2021
(86) PCT No.: PCT/US2021/026919

§ 371 Date: Oct. 7, 2022

Related U.S. Application Data
(60) Provisional application No. 63/009,165, filed on Apr. 13, 2020.
(60) Provisional application No. 63/008,693, filed on Apr. 11, 2020.

(51) Int. Cl. C12Q 1/70; B01L 3/00

(52) U.S. Cl. CPC C12Q 1/701; B01L 3/502; C12Q 1/6851

(57) ABSTRACT

The invention provides an alternative, effective RNA virus sample collection device that samples aerosolized particles from human breath. The invention provides a more direct and medically relevant sample for evaluating transmission risk than a nasopharyngeal swab.

### REFERENCE TO RELATED APPLICATIONS

This invention claims priority under 35 U.S.C. § 119(e) to U.S. Ser. No. 63/008,693, filed Apr. 11, 2020, and titled “A Massively Parallel RNA Virus (COVID-19) Diagnostic Assay,” and U.S. Ser. No. 63/009,165, filed Apr. 13, 2020, and titled “A Massively Parallel RNA Virus (COVID-19) Diagnostic Assay.”

### FIELD OF THE INVENTION

This invention generally relates the measuring or testing processes involving enzymes, nucleic acids, or microorganisms; compositions or test papers therefor; processes of preparing such compositions; condition-responsive control in microbiological or enzymological processes; and to preparing nucleic acids for analysis, e.g., for polymerase chain reaction [PCR] assay.

### BACKGROUND OF THE INVENTION

The COVID-19 virus (SARS-CoV-2) is transmitted through airborne particles in exhaled breath, causing severe respiratory disease. Diagnostic assays for active or prior infection rely on detecting viral RNA or antibodies to the virus. Diagnostic assays are usually performed on patient samples collected from a patient's upper respiratory tract by saliva or nasopharyngeal (NP) swab. These sources have comparable sensitivities, with 97% agreement.

Patient samples for COVID-19 diagnostic assays are frequently collected by nasopharyngeal swab from a patient and detected by a polymerase chain reaction (PCR) in a clinical laboratory. Patients can test positive for three months after infection. This SARS-Cov-2 (COVID-19) diagnostic assay is a time-, reagent-, and labor-intensive protocol that cannot keep pace with current demand. Reagents such as nasopharyngeal (NP) swabs and stabilization solutions are, at many sites, rate-limiting for diagnostic assays by medical clinics.

While upper respiratory tract samples contain the active virus, recent clinical studies have suggested influenza was compartmentalized. Viral loads in the upper respiratory tract, such as the nasal area, were uncorrelated with the lower respiratory tract symptoms, i.e., coughing. Viral loads in aerosolized particles were correlated with the severity of cough symptoms. Because the lower respiratory involvement is often a precursor to more severe COVID outcomes, there is a need in the biomedical art for a more direct sampling approach that focuses on the exhaled breath.

### SUMMARY OF THE INVENTION

The invention provides an alternative, effective RNA virus sample collection device. This sample collection device can replace nasopharyngeal swab, stabilization, RNA extraction, and reverse transcription with a rapid, single step. The collection device samples aerosolized particles from human breath. The invention provides a more direct and medically relevant sample for evaluating transmission risk than a nasopharyngeal swab.

In a first embodiment, the invention provides a device called the Bubbler™, which captures aerosolized RNA-containing particles from a subject's breath. The device is used by having the subject be tested for RNA virus presence, bubbling a breath through an oil/aqueous solution/emulsion contained in the device. In the oil/aqueous solution/emulsion are reagents for carrying out an enzymatic reverse transcriptase (RT) reaction. See FIG. 1(A).

In a second embodiment, the enzyme activity then converts the viral RNA into stable, molecularly barcoded cDNA. The reverse transcriptase activity at the collection site advantageously enables the collection of samples compatible with downstream large-scale sequencing-based parallel diagnostics. See FIG. 3. Alternately, the sample can be diagnosed without sequencing, by PCR.

In a third embodiment, the invention provides a method for reverse transcribing RNA from airborne SARs-CoV-2 viral particles into a sample-specific barcoded cDNA. The method comprises the step of first obtaining a deep breath sample from a patient, such as described in this specification concerning the use of the device of the invention. Next, the sample is reverse transcribed to viral cDNA using sample-specific barcoded primers. Optionally, the samples can then be pooled for analysis by a massively parallel assay.

In a fourth embodiment, the invention provides the device for use as a screen for RNA virus (e.g., COVID-19, SARS, other coronaviruses, influenza viruses, rhinoviruses, or other RNA viruses) in human breath.

In a fifth embodiment, the invention provides the device for use as a screen for RNA virus (e.g., COVID-19, SARS, other coronaviruses, influenza viruses, rhinoviruses, or other RNA viruses) in the environment by applying a vacuum pump to an air vent installed in hospital emergency rooms, airports, building heating, ventilation, and air conditioning (HVAC) air handling systems.

In a sixth embodiment, the invention provides the device for use in detecting a DNA virus in exhaled breath.

In a seventh embodiment, the invention provides the device for use in detecting nonviral nucleic acid in exhaled breath (e.g., DNA from tobacco; DNA from cannabis).

In an eighth embodiment, the invention provides the device for use in detecting aerosolized DNA in the environment.

In a ninth embodiment, the invention provides the device for use in collecting a sample that is used for sequencing to identify the strain of the virus.

In a tenth embodiment, the invention provides the device for use in collecting a sample that is used for [massively parallel assay]

In an eleventh embodiment, the invention provides, the invention provides a device, wherein the receptacle is a balloon, such as a party balloon. RNA virus particles from human breath were readily precipitated from an inflated party balloon's interior surface after a one-hour incubation at −20° C. RT-PCR can readily detect rRNA in this liquid with no RNA extraction.

In one aspect, the invention provides a rapid, high-throughput assay that advantageously enables large-scale survey sequencing. See FIG. 4. The Bubbler™ could even be dispatched for home use, decreasing the current burden on clinical testing facilities.

In another aspect, the invention provides a device and an improved method for determining infectivity. Without an ability to assay SARS-CoV-2 in human breath, a person having ordinary skill in the biomedical art cannot learn the time and circumstance (vaccinated, asymptomatic) when COVID-19 patients are infectious. This situation has been widely appreciated to have contributed to the public uncertainty during the 2020 COVID-19 pandemic.

In another aspect, a diagnosis by sequencing can provide additional information such as viral load and strain identity.

In another aspect, samples from barcode-enabled Bubblers™, where the samples contain cDNA amplified using genetically barcoded primers, can be pooled and batch processed while retaining sample identity.

The invention was tested in a clinical study that demonstrated the feasibility of molecular barcoding coupled with next-generation sequencing to quantitatively detect SARS-CoV-2 in a panel of human-constructed samples at a detection limit of 334 genomic copies/sample.

Tests of the Bubbler™ on seventy patients admitted to hospital showed that it was both more predictive of lower respiratory tract involvement, i.e., abnormal chest X-rays, and less invasive than alternatives. The Bubbler™ sample of exhaled air was three times more enriched for SARS-CoV-2 RNA than tongue swabs. This result implies viral particles were directly sampled.

### BRIEF DESCRIPTION OF THE DRAWINGS

For illustration, some embodiments of the invention are shown in the drawings described below. Like numerals in the drawings indicate like elements throughout the drawings. The invention is not limited to the precise arrangements, dimensions, and instruments shown.

FIG. 1 provides information about the Bubbler™ device. FIG. 1(A) shows the product design. The person being tested exhales through a glass mouthpiece so that aerosolized particles containing viral and cellular RNAs are bubbled through a cool oil/aqueous emulsion. Aerosol particles condense in the aqueous phase and mix with a reverse transcription buffer which copies RNA into barcoded cDNA. FIG. 1(B) shows how a person being tested exhales gently into a hand-held Bubbler™ for less than one minute to evacuate the lungs completely. FIG. 1(C) is a proof of concept. The cellular 18S rRNA was copied into DNA and amplified by PCR. The electrophoresis gel result shows that the Bubbler™ isolates as much RNA in one breath as a conventionally-extracted RNA-labeled control, which was a ˜2-hour Trizol reaction+reverse transcriptase.

FIG. 2 is a diagram showing the massively parallel RNA virus diagnostic assay working on human-constructed samples. (LEFT side) Each device contains a unique barcode appended to the reverse transcription (RT) primer (drawn in purple), adjacent to a random 3-mer (NNN), the reverse primer binding site (labeled Common primer), and a bacteriophage T7 promoter (T7) which is incorporated into the cDNA. The cDNA is treated to remove free primers and protein and then amplified via T7 in vitro transcription. The resulting RNA is reverse transcribed using RT primer 2, amplified by PCR, and analyzed by next-generation sequencing.

FIG. 3 is a diagram that shows an early embodiment of the barcoding/parallelism strategy—showing the massively parallel RNA virus diagnostic assay. This diagram shows the workflow for a single diagnostic performed in parallel on thousands of samples. Step (1): Each Bubbler™ contains a Unique barcode appended to the reverse transcription (RT) primer (drawn in purple), which is incorporated into the cDNA, as shown on the top and middle left. This copying event is the basis of diagnosis as it only occurs if the viral RNA is present in the sample. Step (2): The clinical site returns the kits to a processing center. The contents of the kits are pooled. Step (3): The barcoded cDNAs in the pool are circularized by DNA ligation. Step (4): The circles are amplified by inverted PCR primers and analyzed by next-generation sequencing. The bottom right describes the sequence analysis. The presence of barcodes (purple) is associated with a positive test result. The number of times a barcode is sequenced is proportional to the viral load. The (blue) copied viral sequences contain viral strain information useful in reconstructing transmission paths.

FIG. 4 is a diagram showing a diagnostic matrix returned by an assay. Each kit contains an ensemble of uniquely barcoded RT primers specific to at least twenty-seven different RNAs. These RNAs are targeted to respiratory pathogens and human RNAs of varying abundance and various cellular origins. The assay identifies the pathogen and the quality of the sample.

FIG. 5 shows a view of the Bubbler™ device with several permutations for several uses. FIG. 5(A) For environmental sampling, a vacuum line is attached to the air vents to draw a continuous airflow through the Bubbler™. FIG. 5(B) The device can be miniaturized to a tube size compatible with liquid handling platforms. FIG. 5(C) Canola oil, mineral oil (any non-toxic oil). FIG. 5(D) The solution can be H2O, TE solution, a readily substitutable replacement solution. For environmental sampling, DNAzol™ RNAzol™, phenol/chloroform 1:1 solution, H2O, TE solution can be used.

FIG. 6 shows a molecular characterization of the bubblers sample relative to alternate (tongue scrape, saliva) sampling technology. RT-PCR demonstrates the presence of cellular RNA (18S, bottom panel) but the absence of ACE2R (COVID-19 viral receptor). This finding supports the idea that the COVID-19 signal from the bubbler comes predominantly from VIRAL PARTICLES and not viral transcripts in infected cells.

FIG. 7 shows the implementation on a contrived COVID sample panel. The panel consists of ten serial 5-fold dilutions of a COVID standard (ATCC, VR-1986D, Lot 70035624) arrayed in a manner prescribed by the FDA emergency use authorization guidelines. Panel A shows the RT primer described in FIG. 2. Panel B shows the dilution scheme used to calculate a detection limit of 334 viral particles/breath.

### DETAILED DESCRIPTION OF THE INVENTION

### Industrial Applicability

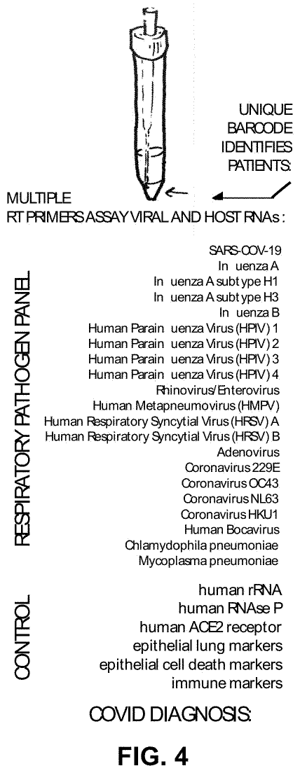

The emergence of COVID in 2019 revealed the need for improvements in the modern response to sudden pandemics. To slow or stop the spread of the COVID-19 virus and other airborne viruses, especially airborne RNA viruses such as influenza viruses, coronaviruses, and rhinoviruses (see FIG. 4), must (a) know who is infected and (b) be able to test many people at once. During the 2019 COVID pandemic, testing was often limited for different reasons. Initial problems with establishing a reliable diagnostic gave way to a lack of capacity at diagnostic labs and eventual shortages in reagents to run diagnostic tests. While COVID cases are declining in 2021, the need for mass testing is still strong. This need could be exacerbated if a vaccine-resistant strain emerges.

The Bubbler™ (which in Rhode Island and some other places can be called the Buhblah™) is an attractive alternative to current swab-based sample collection technologies. The Bubbler™ can replace the nasopharyngeal swab, stabilization, RNA extraction, and reverse transcription with a rapid, single step. This collection device samples aerosolized particles from human breath is a more direct and medically relevant sample for evaluating the risk of transmission than a nasopharyngeal swab. This hand-held device captures aerosolized RNA containing particles from breath by bubbling through an oil/aqueous emulsion that contains enzymatic reverse transcriptase activity, which converts viral RNA into stable, molecularly barcoded cDNA. A key advance is including the RT step at the collection site as this enables the collection of samples compatible with large-scale downstream sequencing-based parallel diagnostics.

Several devices were designed to capture exhaled breath condensate. Breathalyzers have been developed to sample metabolites. Prior studies failed to detect differences between the lung microbiome and the microbiome of the upper respiratory tract. See Charlson et al., Am. J. Respir. Crit. Care Med. (184), 957-963 (2011). However, some cellular genes are expressed predominantly in the lung, such as the family of Surfactant-associated proteins (e.g., SP-A). ACE-2 expression is found but not restricted to the lung. Hermans &. Bernard, Am. J. Respir. Crit. Care Med. 159, 646-678 (1999).

The invention provides a device and method orthogonal to existing COVID-19 testing protocols. The parallelism could be expanded to include multiple tests or an entire respiratory panel in one Bubbler™ so diseases with similar symptoms can be tested jointly.

The device and method enable testing tens of thousands of people a day more conveniently and comfortably than taking a nasal swab.

The rapid high-throughput assay enables large-scale survey sequencing. The Bubbler™ can be dispatched for home use, decreasing the burden on current testing facilities.

The invention described in this specification does not concern a process for cloning humans, processes for modifying the germ line genetic identity of humans, uses of human embryos for industrial or commercial purposes, or processes for modifying the genetic identity of animals likely to cause them suffering with no substantial medical benefit to man or animal, and also animals resulting from such processes.

### Definitions

For convenience, the meaning of some terms and phrases used in the specification, examples, and appended claims, are listed below. Unless stated otherwise or implicit from context, these terms and phrases shall have the meanings below. These definitions aid in describing particular embodiments but are not intended to limit the claimed invention. Unless otherwise defined, all technical and scientific terms have the same meaning as commonly understood by a person having ordinary skill in the art to which this invention belongs. A term's meaning provided in this specification shall prevail if any apparent discrepancy arises between the meaning of a definition provided in this specification and the term's use in the biomedical art.

Unless otherwise defined herein, scientific and technical terms used with this application shall have the meanings commonly understood by persons having ordinary skill in the biomedical art. This invention is not limited to the particular methodology, protocols, and reagents, etc., described herein and as such can vary.

“About” has the plain meaning of approximately. The term about encompasses the measurement errors inherently associated with the relevant testing. When used with percentages, about means ±1%.

“Air vent” has the plain meaning of an opening that allows air to pass out of or into a confined space. Air vents that could contain airborne viruses are installed in hospital emergency rooms, airports, and building heating, ventilation, and air conditioning (HVAC) aft handling systems.

“Airborne” has the plain meaning of a particle that is traveling in the air. Early research from different types of the exhaled breath (e.g., sneezing, coughing, and talking loudly) has demonstrated a wide range of droplet sizes that persist in the air. The smaller drops persist longer. The larger droplets reduce to smaller droplets through evaporation. Culturing droplets for the commensurate Str. viridans illustrated how pathogens could travel within aerosolized droplets in exhaled breath. These studies concluded that 90% of airborne bacteria could persist in droplets for thirty-sixty minutes in unventilated space. Smaller viruses could presumably persist longer and travel further.

“Alert Level” is an established microbial or airborne particle level giving early warning of potential drift from normal operating conditions and triggers appropriate scrutiny and follow-up to address the potential problem. See United States Food and Drug Administration, Guidance for Industry, Sterile Drug Products Produced by Aseptic Processing—Current Good Manufacturing Practice (September 2004).

“Aseptic Processing Facility” is a building, or segregated segment of it, containing cleanrooms in which air supply, materials, and equipment are regulated to control microbial and particle contamination. See, United States Food and Drug Administration, Guidance for Industry, Sterile Drug Products Produced by Aseptic Processing—Current Good Manufacturing Practice (September 2004).

“Clean area” or a “clean zone” is an area with defined particle and microbiological cleanliness standards. See United States Food and Drug Administration, Guidance for Industry, Sterile Drug Products Produced by Aseptic Processing—Current Good Manufacturing Practice (September 2004).

“Coronavirus” has the biomedical art-recognized meaning of the group of related RNA viruses that cause diseases in mammals and birds. Coronaviruses constitute the subfamily Orthocoronavirinae, in the family Coronaviridae, order Nidovirales, and realm Riboviria. They are enveloped viruses with a positive-sense single-stranded RNA genome and a nucleocapsid of helical symmetry.

“COVID-19” (SARS-CoV-2) is a coronavirus that gains entry into a wide range of cell types through the ACE2 receptor and causes COVID-19, a severe respiratory disorder. COVID-19 is characterized by fever, a dry cough, and a variety of other symptoms. While COVID-19 can present with symptoms outside the lower respiratory tract, a dangerous trajectory can cause inflammation in the lungs resulting in pneumonia. Because SARS-CoV-2 is an airborne pathogen, the infection status of the lungs and airway is predictive not only of disease outcome but also the risk of transmission.

“DNA virus” has the biomedical art-recognized definition of a virus whose genetic material is deoxyribonucleic acid. There are six generally-recognized classes of viruses. The DNA viruses constitute classes I (double stranded DNA viruses) and II (single-stranded DNA viruses).

“Environment” has the plain meaning of the surroundings or conditions in which a person, animal, or plant lives or operates, especially the surrounding air.

“McNemar's test” has the statistical art-recognized meaning. In statistics, McNemar's test is a statistical test used on paired nominal data. It is applied to 2×2 contingency tables with a dichotomous trait, with matched pairs of subjects, to determine whether the row and column marginal frequencies are equal (that is, whether there is “marginal homogeneity”).

“Nucleic acid” and “nucleic acid molecule” can be used interchangeably herein and refer to a polymer or polymer block of nucleotides or nucleotide analogues. The nucleic acid may be obtained from natural sources or may be produced recombinantly or by chemical synthesis. The nucleic acid can be single, double, or multiple stranded and may comprise modified or unmodified nucleotides or non-nucleotides or various mixtures and combinations thereof.

“Patient” to refer to any person to whom the assay is administered. In particular, a patient can refer to a person who has or is suspected of having COVID-19, to whom the assay is administered. The terms “patient,” “individual,” and “subject” are interchangeable.

“Polymerase chain reaction” (PCR) has the biomedical art-recognized meaning of a method widely used to rapidly make millions to billions of copies of a specific DNA sample, allowing scientists to take a very small sample of DNA and amplify it to a large enough amount to study in detail. Using PCR, copies of very small amounts of DNA sequences are exponentially amplified in a series of cycles of temperature changes. PCR is now a common and often indispensable technique used in medical laboratory research for a broad variety of applications including biomedical research and criminal forensics. PCR kits are commercially available.

“Respiratory disease” has the biomedical art-recognized definition. Common viral respiratory diseases are illnesses caused by a variety of viruses that have similar traits and affect the respiratory tract. The viruses involved may be the influenza viruses, respiratory syncytial virus (RSV) (the major cause of bronchiolitis, pneumonia, croup, bronchitis, and otitis media), parainfluenza viruses (the major cause of croup in young children and can cause bronchitis, pneumonia, and bronchiolitis), or respiratory adenoviruses (which can cause a variety of illnesses from pharyngitis to pneumonia, conjunctivitis, and diarrhea). Other viruses include rhinoviruses (which typically causes the common cold) and coronaviruses. Infection with viruses in the respiratory tract can cause complications such as tonsillitis, laryngitis, bronchitis, pneumonia. See Boncristiani, Respiratory viruses. Encyclopedia of Microbiology, 500-518 (Feb. 17, 2009).

“Reverse transcriptase” (RT) has the biomedical art-recognized meaning of an enzyme that catalyzes the formation of DNA from an RNA template in reverse transcription. Reverse transcriptase is commercially available.

“Ribonucleic acid” (RNA) has the biomedical art-recognized meaning of a ribose-containing nucleic acid. Its principal role is to act as a messenger carrying instructions from DNA for controlling the synthesis of proteins, although in some viruses RNA rather than DNA carries the genetic information.

“RNA virus” has the biomedical art-recognized definition of a virus whose genetic material is ribonucleic acid. The RNA may be either double- or single-stranded. There are six generally-recognized classes of viruses. The DNA viruses constitute classes I and II. The RNA viruses make up the remaining classes. Class III viruses have a double-stranded RNA genome. Class IV viruses have a positive single-stranded RNA genome, the genome itself acting as mRNA (messenger RNA. Class V viruses have a negative single-stranded RNA genome used as a template for mRNA synthesis. Class VI viruses have a positive single-stranded RNA genome but with a DNA intermediate not only in replication but also in mRNA synthesis. Notable human respiratory diseases caused by RNA viruses include the common cold, influenza, SARS, MERS, and COVID-19.

Cochrane-Armitage test has the statistical art-recognized meaning. The Cochran-Armitage test for trend is used in categorical data analysis when the aim is to assess for the presence of an association between a variable with two categories and an ordinal variable with k categories. It modifies the Pearson chi-squared test to incorporate a suspected ordering in the effects of the k categories of the second variable.

Guidance from Materials and Methods

A person having ordinary skill in the art can use these materials and methods as guidance to predictable results when making and using the invention:

### The Bubbler™—Instructions for Use

The Bubbler™ is a hand-held device with a glass straw at the top into which the subject being tested blows a breath. The glass straw can be made from a Pasteur pipette. The device is a breathalyzer for viruses. This device can be used to determine who is infected with a respiratory virus, such as an RNA virus, such as an influenza virus, rhinovirus, or coronavirus, such as COVID-19. See FIG. 4. The device enables a person having ordinary skill in the medical art to test tens of thousands of people a day so it is easier, more convenient, and more comfortable than taking a nasal swab.

The person being tested shall take these steps:

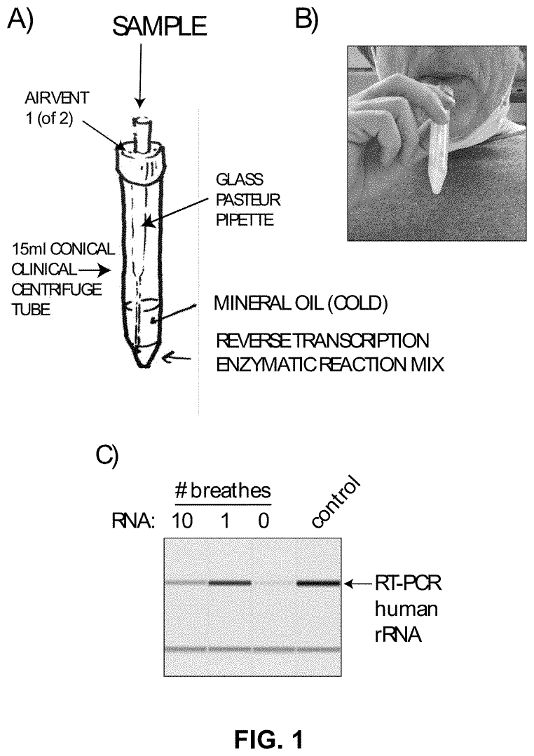

Step (1). Hold the Bubbler™ from the top. See FIG. 1(B).

Step (2). Tilt slightly down and take a normal breath.

Step (3). Exhale into the tube.

The person being tested or the person performing the test should hear a bubbling sound. The person being tested shall blow out all the air in the lungs. This process should take no more than 10 seconds. The oil/water mix at the bottom of the tube should be an emulsion (like oil and vinegar salad dressing). Sometimes a little saliva gets into the Bubbler™, so the last step is to get a saliva sample.

### Methods for Clinical Study of Efficacy of Massively Parallel RNA Virus Diagnostic Assay

Enrollment of study participants and sample collection. The clinical staffed screened the clinical study participants in a hospital from May 2020-January 2021, during the COVID-19 pandemic. The eligible patients were over 18 years old, had COVID-19 testing collected or historically available within seventy-two hours, spoke English, and understood and provided written informed consent. Patients unable to provide informed consent as determined by the clinical providers were excluded. From each enrolled subject, the inventors collected ˜fifteen seconds of exhaled breath in the Bubbler™ as well as two tongue scrapings. After ˜thirty minutes at room temperature, samples were transferred to −80° C. until laboratory testing.

Clinical study sample preparation, PCR and real-time PCR. SuperScript™ IV reverse transcriptase (Thermo Fisher, 18090050) was mixed with reverse transcription (RT) primer and dNTP to make a 40 μl reaction for the Bubbler™ or a 20 μl reaction for the tongue scrape. Eight primers for Sars-Cov-2 N gene and one primer for RNaseP (see sequences in SEQUENCE LISTING) are pooled to the concentration of 20 μM and used as a RT primer pool. 0.5 μl RT mix from patient sample is mixed with primers (listed in the SEQUENCE LISTING) and Power SYBR Green PCR Master Mix (Thermo Fisher, 4367659) to make a 10 μl reaction for real-time PCR analysis. The real-time PCR program is set as: (1) hold stage: 50° C. for two minutes, then 95° C. for three minutes; (2) PCR: 95° C. for fifteen seconds, 60° C. for twenty seconds and 72° C. for thirty seconds, 40 cycles; 3). Melt curve: 95° C. at fifteen seconds, 60° C. for twenty seconds, then increase to 95° C. with the speed of 0.05° C./s, hold at 95° C. for fifteen seconds. When Ct<35 for both real-time PCR primer sets, the patient sample is determined as positive for Sars-Cov-2. GoTaq Master Mix (Promega, M7123) is used in PCR reaction to detect 18S rRNA or ACE2. See the sequences of the primers in the SEQUENCE LISTING. Human total RNA (Thermo Fisher, 4307281, Lot 00890901) and SARS-CoV-2 genomic RNA (ATCC, VR-1986D, Lot 70035624) are used as controls.

Quantitative polymerase chain reaction (qPCR). The copy number of SARS-CoV-2 N gene RNA used in human-constructed samples was quantified by qPCR. RNA was reverse transcribed to cDNA via SuperScript™ IV transcriptase (Thermo Fisher Scientific, Cat #18090050) using random 9-mer. The resulting cDNA was added to the qPCR reaction using Applied Biosystems Power SYBR™ Green PCR Master Mix (Thermo Scientific). In vitro transcribed N gene RNA were used to prepare absolute standards. The inventors then generated a standard curve to calculate copy number. qPCR reaction was performed on ViiA 7 Real-time PCR Systems using CDC N1 SARS-CoV-2 primer.

Statistical Analysis of the Diagnostic Tests. To compare clinical usefulness of the Bubbler™ PCR method (B-PCR), Hospital PCR (H-PCR) and Laboratory PCR (L-PCR) were categorized as positive (POS) or negative (NEG). The L-PCR was also duplicated to confirm its result; a POS result was assigned if either of the two tests were positive. Radiographic findings (XR) were also dichotomized as normal or abnormal based on any radiographic signs of viral pneumonia. Agreement between H-PCR and L-PCR, B-PCR and H-PCR measures was assessed using 2×2 tables (SAS version 9.4 proc freq) to evaluate the proportion of patients who were categorized as POS by gold standard L-PCR versus H-PCR or B-PCR, and in proportion of patients H-PCR POS who were also XR POS. The sensitivity, specificity, and PPV values were also reported as indicators of the usefulness of the B-PCR in predicting COVID-19 positivity. The L-PCR was the comparison standard in this EXAMPLE and not H-PCR. A POS Bubbler™ result are Bubblers™ that were POS from patients that were also POS on either of the duplicated L-PCR assays. A NEG Bubbler™ result are Bubblers™ that were NEG from patients that were also NEG on either of the duplicated L-PCR assays.

McNemar's test was used in all of the dichotomized comparisons. Estimates were reported with 95% CI's. Estimates were then rank-ordered from to least to most positive and tested using the Cochrane-Armitage test for trend, using one-tailed hypothesis testing, to determine if the rates of abnormal chest XR results are predicted by B-PCR.

To analyze the difference in relative SARS-CoV-2 expression between the tongue scrape and Bubbler™ tests, a subset of the data was taken that contained only positive test results. Using the comparative CT method, the CT numbers for SARS-CoV-2 amplification were converted to their relative expression levels compared to the RNase P control in the sample. The median values of these relative expressions were calculated separately for both the tongue scrape and the Bubbler™. Several successive tests were performed after excluding outliers in the data. For each test, the median relative expression of SARS-CoV-2 was larger (t-test) for the Bubbler™ than for the tongue scrape (see Table S4).

In vitro RNA transcription. DNA oligonucleotide of SARS-CoV-2 N gene with a T7 promoter was synthesized at Integrated DNA Technologies (IDT). PCR amplification was performed on this oligonucleotide using Q5 High fidelity DNA polymerase (NEB) to prepare a template for in vitro transcription (IVT). Primers were listed in Table 51. A single PCR amplicon was confirmed by agarose gel electrophoresis. IVT was performed using Riboprobe® System-T7 kit (Promega, Cat #P1440) following the manufacture's recommendations. The DNA template was removed by digestion with DNase I, and IVT RNA was subsequently extracted using phenol (pH4.7):chloroform and precipitated by ethanol.

High throughput testing on human-constructed samples. A five-fold serial dilution using the IVT N gene RNA was performed in triplicate to make human-constructed samples. Ten dilutions and two blank controls were included in each replicate. Human total RNA control (Thermo Fisher Scientific, Cat #43-072-81) was used as diluent. Barcoded transcript-specific RT primers were synthesized in a 96-well plate at IDT. Each barcoded primer contains a targeting region either binding to human 18S rRNA or N gene, a 3 nucleotide random sequence (Unique Molecular Identifiers, UMI), a 8 nucleotide barcode, a constant region where PCR reverse primer binds and a T7 promoter. FIG. 4(A). See the SEQUENCE LISTING for primers. Thirty-six human-constructed samples were arrayed into 96-well where barcoded RT primers were already assigned, and each well contained two barcoded RT primers, one for 18S rRNA and the other for N gene RNA. RNA was then reverse transcribed to double-stranded cDNA via Maxima H Minus Double-Stranded cDNA Synthesis Kit (Thermo Fisher Scientific, Cat #K2561) following manufacturers recommendations. Residual RNA and RT primers were removed with RNase I and exonuclease I, respectively. After Proteinase K treatment, all the cDNA were pooled and purified using QIAquick PCR Purification kit (Qiagen, Cat #28004), then underwent in vitro transcription reaction. The resulting antisense RNA was then reverse transcribed to cDNA via SuperScript™ IV transcriptase (Thermo Fisher Scientific, Cat #18090050) using specific RT primers both for 18S rRNA and N gene. The following two step nested PCR amplification uses the same reverse primer and two different forward primers. Specific RT and PCR primers are listed in the SEQUENCE LISTING. Amplicon sequencing was performed to quantify each barcode.

Analysis of human-constructed sample amplicon sequencing. The common reverse primer (RP) sequence used in the serial dilutions described was mapped to the reads obtained from amplicon sequencing using bowtie2. The sample barcode and UMI were obtained from the adjacent sequence for reads containing the full-length RP. These reads were then trimmed of non-target sequence (i.e. the UMI, sample barcode and RP) and mapped to the targeted sequence (either SARS-CoV-2 or 18S rRNA) to confirm that they contain the expected sequence between the forward primer (either FP1 or FP2) and the 1^{st} round reverse transcription primer. Read counts for each dilution level's barcode were calculated from the set of reads that contained the expected RP and target sequence. As each dilution level should contain five-fold less SARS-CoV-2 RNA as the previous level, the expected read count for a given dilution level is set to one fifth the number of reads observed from the previous level. The expectation for the read counts of the two water-based blank samples was taken to be zero. The expected read count for the first dilution level was set to the observed read count for plotting purposes, but this level was excluded from correlation calculations. The Pearson correlation coefficient was calculated for these comparisons: observed FP1 counts vs. observed FP2 counts, observed FP1 counts vs. expected FP1 counts, and observed FP2 counts vs. expected FP2 counts.

TABLE 1
Statistical comparison of COVID-19 tests

| Statistic Estimate | H-PCR vs. | H-PCR vs. | H-PCR vs. | B-PCR vs. |
|---|---|---|---|---|
| (95% CI) | L-PCR | X-Ray | B-PCR | X-Ray |
| Sensitivity | 0.94 | 0.66 | 0.89 | 0.50 |
| | (0.82, 1.0) | (0.49, 0.82) | (0.74, 1.0) | (0.33, 0.67) |
| Specificity | 0.80 | 0.95 | 0.82 | 0.96 |
| | (0.68, 0.93) | (0.87, 1.0) | (0.70, 0.94) | (0.87, 1.0) |
| Positive | 0.65 | 0.95 | 0.69 | 0.94 |
| Predictive | (0.46, 0.85) | (0.86, 1.0) | (0.51, 0.88) | (0.82, 1.0) |
| Value | | | | |
| Negative | 0.97 | 0.67 | 0.94 | 0.58 |
| Predictive | (0.91, 1.0) | (0.51, 0.83) | (0.86, 1.0) | (0.42, 0.74) |
| Value | | | | |
| McNemar’s test | x₁² = 5.42, | x₁² = 8.33, | x₁² = 2.78, | x₁² = 13.2, |
| | p = 0.02 | p = 0.01 | p = 0.10 | p = 0.001 |

Analysis based on n = 57

In vitro RNA transcription. DNA oligonucleotide of SARS-CoV-2 N gene with a T7 promoter was synthesized at Integrated DNA Technologies (IDT). PCR amplification was performed on this oligonucleotide using Q5 High fidelity DNA polymerase (NEB) to prepare a template for in vitro transcription (IVT). Primers are listed in the SEQUENCE LISTING. A single PCR amplicon was confirmed by agarose gel electrophoresis.

In vitro RNA transcription was performed using Riboprobe® System-T7 kit (Promega, Cat #P1440) following the manufacture's recommendations. The DNA template was removed by digestion with DNase I, and transcribed RNA was subsequently extracted using phenol (pH4.7):chloroform and precipitated by ethanol.

### In Vitro RNA Transcription

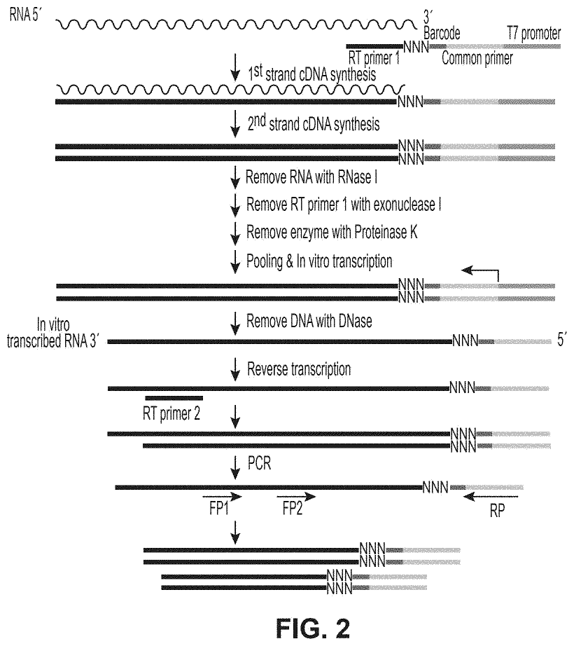

High throughput testing on human-constructed samples. A five-fold serial dilution using the in vitro transcribed N gene RNA was performed in triplicate to make human-constructed samples. Ten dilutions and 2 blank controls were included in each replicate. Human total RNA control (Thermo Fisher Scientific, Cat #43-072-81) was used as diluent. Barcoded transcript-specific RT primers were synthesized in a 96-well plate at Integrated DNA Technologies. Each barcoded primer contains a targeting region either binding to human 18S rRNA or N gene, a 3-nucleotide randomer (Unique Molecular Identifiers, UMI), a 8-nucleotide barcode, a constant region where PCR reverse primer binds and a T7 promoter. See the SEQUENCE LISTING for primers. Thirty-six human-constructed samples were arrayed into 96-well where barcoded RT primers were already assigned and each well contained 2-barcoded RT primers, one for 18S rRNA and the other for N gene RNA. Then RNA was reverse transcribed to double-stranded cDNA via Maxima H Minus Double-Stranded cDNA Synthesis Kit (Thermo Fisher Scientific, Cat #K2561) following manufacturer's recommendations. Residual RNA and RT primers were removed with RNase I and exonuclease I, respectively. See FIG. 2. After the Proteinase K treatment, all the cDNA were pooled and purified using QIAquick PCR Purification kit (Qiagen, Cat #28004), then underwent in vitro transcription reaction. The resulting antisense RNA was then reverse transcribed to cDNA via SuperScript™ IV transcriptase (Thermo Fisher Scientific, Cat #18090050) using specific RT primers both for 18S rRNA and N gene. The following two step nested PCR amplification uses the same reverse primer and two different forward primers. Specific RT and PCR primers were listed in the SEQUENCE LISTING. Amplicon sequencing was performed to quantify each barcode.

SEQUENCE LISTING
Primers used in the clinical study
RT primers
hRPp1_RT-
(SEQ ID NO: 1)
NNNNNNGAATTGGGTTA.
Cv_RT1-
(SEQ ID NO: 2)
NNNNNNCAGCACTGCTC.
Cv_RT2-
(SEQ ID NO: 3)
NNNNNNCCTGAGTTGAG.
Cv_RT3-
(SEQ ID NO: 4)
NNNNNNAGTTGAGTCAG.
Cv_RT4-
(SEQ ID NO: 5)
NNNNNNAGTCAGCACTG.
Cv_RT5-
(SEQ ID NO: 6)
NNNNNNGAGTCAGCACT.
Cv_RT6-
(SEQ ID NO: 7)
NNNNNNGTTGAGTCAGC.
Cv_RT7-
(SEQ ID NO: 8)
NNNNNNGGCCTGAGTTG.
Cv_RT8-
(SEQ ID NO: 9)
NNNNNNGTCAGCACTGC.
PCR primers
18S_FP-
(SEQ ID NO: 10)
TGCAATTATTCCCCATGAACGAG.
18S_RP-
(SEQ ID NO: 11)
CTAGATAGTCAAGTTCGACCGTC.
ACE2_FP-
(SEQ ID NO: 12)
TTCGGCTTCGTGGTTAAACT.
ACE2_RP-
(SEQ ID NO: 13)
CTCTTCCTGGCTCCTTCTCA.
Real-time PCR primers
hRPp1_rtPCR_1-
(SEQ ID NO: 14)
GGATGCCTCCTTTGCCGGAG.
hRPp1_rtPCR_2-
(SEQ ID NO: 15)
AGCCATTGAACTCACTTCGC.
Cv19N_rtPCR1_1-
(SEQ ID NO: 16)
AGTCAAGCCTCTTCTCGTTCC.
Cv19N_rtPCR1_2-
(SEQ ID NO: 17)
GCAAAGCAAGAGCAGCATCAC.
Cv19N_rtPCR2_1-
(SEQ ID NO: 18)
GGTGTTAATTGGAACGCCTTGTCCTC.
Cv19N_rtPCR2_2-
(SEQ ID NO: 19)
TCTTGGTTCACCGCTCTCACTCA.
Primers used for human-constructed sample
testing. The RT primer 1 in FIG. 2 refers to
from N-gene-BC1 to N-gene-BC36.
N-gene-BC1-
(SEQ ID NO: 20)
TAATACGACTCACTATAGGGCCGATATCCGACGGTAGTGTCACGTCG
TNNNATCATCCAAATCTGCAG.
N-gene-BC2-
(SEQ ID NO: 21)
TAATACGACTCACTATAGGGCCGATATCCGACGGTAGTGTCAATTGA
TNNNATCATCCAAATCTGCAG.
N-gene-BC3-
(SEQ ID NO: 22)
TAATACGACTCACTATAGGGCCGATATCCGACGGTAGTGTATATTGTA
NNNATCATCCAAATCTGCAG.
N-gene-BC4-
(SEQ ID NO: 23)
TAATACGACTCACTATAGGGCCGATATCCGACGGTAGTGTATAGCAC
GNNNATCATCCAAATCTGCAG.
N-gene-BC5-
(SEQ ID NO: 24)
TAATACGACTCACTATAGGGCCGATATCCGACGGTAGTGTACACATG
TNNNATCATCCAAATCTGCAG.
N-gene-BC6-
(SEQ ID NO: 25)
TAATACGACTCACTATAGGGCCGATATCCGACGGTAGTGTATGTAAT
GNNNATCATCCAAATCTGCAG.
N-gene-BC7-
(SEQ ID NO: 26)
TAATACGACTCACTATAGGGCCGATATCCGACGGTAGTGTAGTATCT
GNNNATCATCCAAATCTGCAG.
N-gene-BC8-
(SEQ ID NO: 27)
TAATACGACTCACTATAGGGCCGATATCCGACGGTAGTGTATGCTTG
ANNNATCATCCAAATCTGCAG.
N-gene-BC9-
(SEQ ID NO: 28)
TAATACGACTCACTATAGGGCCGATATCCGACGGTAGTGTAACTGTA
TNNNATCATCCAAATCTGCAG.
N-gene-BC 10-
(SEQ ID NO: 29)
TAATACGACTCACTATAGGGCCGATATCCGACGGTAGTGTCAGGCA
TTNNNATCATCCAAATCTGCAG.
N-gene-BC11-
(SEQ ID NO: 30)
TAATACGACTCACTATAGGGCCGATATCCGACGGTAGTGTAAGGCG
ATNNNATCATCCAAATCTGCAG.
N-gene-BC12-
(SEQ ID NO: 31)
TAATACGACTCACTATAGGGCCGATATCCGACGGTAGTGTGCGTCG
AANNNATCATCCAAATCTGCAG.
N-gene-BC13-
(SEQ ID NO: 32)
AATACGACTCACTATAGGGCCGATATCCGACGGTAGTGTGAACGAC
ANNNATCATCCAAATCTGCAG.
N-gene-BC14-
(SEQ ID NO: 33)
TAATACGACTCACTATAGGGCCGATATCCGACGGTAGTGTGGCAAG
CANNNATCATCCAAATCTGCAG.
N-gene-BC15-
(SEQ ID NO: 34)
TAATACGACTCACTATAGGGCCGATATCCGACGGTAGTGTGTAACC
CANNNATCATCCAAATCTGCAG.
N-gene-BC16-
(SEQ ID NO: 35)
TAATACGACTCACTATAGGGCCGATATCCGACGGTAGTGTGCTATG
CANNNATCATCCAAATCTGCAG.
N-gene-BC17-
(SEQ ID NO: 36)
TAATACGACTCACTATAGGGCCGATATCCGACGGTAGTGTGACACT
TANNNATCATCCAAATCTGCAG.
N-gene-BC18-
(SEQ ID NO: 37)
TAATACGACTCACTATAGGGCCGATATCCGACGGTAGTGTGGTTGG
ACNNNATCATCCAAATCTGCAG.
N-gene-BC19-
(SEQ ID NO: 38)
TAATACGACTCACTATAGGGCCGATATCCGACGGTAGTGTTCAGATT
CNNNATCATCCAAATCTGCAG.
N-gene-BC20-
(SEQ ID NO: 39)
TAATACGACTCACTATAGGGCCGATATCCGACGGTAGTGTTATGCC
AGNNNATCATCCAAATCTGCAG.
N-gene-BC21-
(SEQ ID NO: 40)
TAATACGACTCACTATAGGGCCGATATCCGACGGTAGTGTTGGCTC
AGNNNATCATCCAAATCTGCAG.
N-gene-BC22-
TAATACGACTCACTATAGGGCCGATATCCGACGGTAGTGTTCATTGA
CNNNATCATCCAAATCTGCAG.
(SEQ ID NO: 41)
N-gene-BC23-
(SEQ ID NO: 42)
TAATACGACTCACTATAGGGCCGATATCCGACGGTAGTGTTGTATG
CGNNNATCATCCAAATCTGCAG.
N-gene-BC24-
(SEQ ID NO: 43)
TAATACGACTCACTATAGGGCCGATATCCGACGGTAGTGTTCCAGT
CGNNNATCATCCAAATCTGCAG.
N-gene-BC25-
TAATACGACTCACTATAGGGCCGATATCCGACGGTAGTGTTACTTC
CGNNNATCATCCAAATCTGCAG.
(SEQ ID NO: 44)
N-gene-BC26-
(SEQ ID NO: 45)
TAATACGACTCACTATAGGGCCGATATCCGACGGTAGTGTTGAACT
CGNNNATCATCCAAATCTGCAG.
N-gene-BC27-
(SEQ ID NO: 46)
TAATACGACTCACTATAGGGCCGATATCCGACGGTAGTGTTTGGTAT
CNNNATCATCCAAATCTGCAG.
N-gene-BC28-
(SEQ ID NO: 47)
TAATACGACTCACTATAGGGCCGATATCCGACGGTAGTGTTAACGC
TGNNNATCATCCAAATCTGCAG.
N-gene-BC29-
(SEQ ID NO: 48)
TAATACGACTCACTATAGGGCCGATATCCGACGGTAGTGTTTCCATT
CNNNATCATCCAAATCTGCAG.
N-gene-BC30-
(SEQ ID NO: 49)
TAATACGACTCACTATAGGGCCGATATCCGACGGTAGTGTTGTGGT
TGNNNATCATCCAAATCTGCAG.
N-gene-BC31-
(SEQ ID NO: 50)
TAATACGACTCACTATAGGGCCGATATCCGACGGTAGTGTTACAGG
ATNNNATCATCCAAATCTGCAG.
N-gene-BC32-
(SEQ ID NO: 51)
TAATACGACTCACTATAGGGCCGATATCCGACGGTAGTGTTTCCTG
CTNNNATCATCCAAATCTGCAG.
N-gene-BC33-
(SEQ ID NO: 52)
TAATACGACTCACTATAGGGCCGATATCCGACGGTAGTGTTGCGAT
CTNNNATCATCCAAATCTGCAG.
N-gene-BC34-
(SEQ ID NO: 53)
TAATACGACTCACTATAGGGCCGATATCCGACGGTAGTGTTGCATA
CTNNNATCATCCAAATCTGCAG.
N-gene-BC35-
(SEQ ID NO: 54)
TAATACGACTCACTATAGGGCCGATATCCGACGGTAGTGTTGATAC
CTNNNATCATCCAAATCTGCAG.
N-gene-BC36-
(SEQ ID NO: 55)
TAATACGACTCACTATAGGGCCGATATCCGACGGTAGTGTTCGAGC
GTNNNATCATCCAAATCTGCAG.
18S-rRNA-BC1-
(SEQ ID NO: 56)
TAATACGACTCACTATAGGGCCGATATCCGACGGTAGTGTGCTTC
ACANNNGACGGGCGGTGTGTAC.
18S-rRNA-BC2-
(SEQ ID NO: 57)
TAATACGACTCACTATAGGGCCGATATCCGACGGTAGTGTCGATG
TTTNNNGACGGGCGGTGTGTAC.
18S-rRNA-BC3-
(SEQ ID NO: 58)
TAATACGACTCACTATAGGGCCGATATCCGACGGTAGTGTTTAGG
CATNNNGACGGGCGGTGTGTAC.
18S-rRNA-BC4-
(SEQ ID NO: 59)
TAATACGACTCACTATAGGGCCGATATCCGACGGTAGTGTACAGT
GGTNNNGACGGGCGGTGTGTAC.
18S-rRNA-BC5-
(SEQ ID NO: 60)
TAATACGACTCACTATAGGGCCGATATCCGACGGTAGTGTGCCAA
TGTNNNGACGGGCGGTGTGTAC.
18S-rRNA-BC6-
(SEQ ID NO: 61)
TAATACGACTCACTATAGGGCCGATATCCGACGGTAGTGTCAGAT
CTGNNNGACGGGCGGTGTGTAC.
18S-rRNA-BC7-
(SEQ ID NO: 62)
TAATACGACTCACTATAGGGCCGATATCCGACGGTAGTGTACTTG
ATGNNNGACGGGCGGTGTGTAC.
18S-rRNA-BC8-
(SEQ ID NO: 63)
TAATACGACTCACTATAGGGCCGATATCCGACGGTAGTGTTAGCT
TGTNNNGACGGGCGGTGTGTAC.
18S-rRNA-BC9-
(SEQ ID NO: 64)
TAATACGACTCACTATAGGGCCGATATCCGACGGTAGTGTTGGTT
GTTNNNGACGGGCGGTGTGTAC.
18S-rRNA-BC10-
(SEQ ID NO: 65)
TAATACGACTCACTATAGGGCCGATATCCGACGGTAGTGTTGTAC
GTTNNNGACGGGCGGTGTGTAC.
18S-rRNA-BC11-
(SEQ ID NO: 66)
TAATACGACTCACTATAGGGCCGATATCCGACGGTAGTGTTCTG
CTGTNNNGACGGGCGGTGTGTAC.
18S-rRNA-BC12-
(SEQ ID NO: 67)
TAATACGACTCACTATAGGGCCGATATCCGACGGTAGTGTTTGG
AGGTNNNGACGGGCGGTGTGTAC.
(SEQ ID NO: 68)
18S-rRNA-BC13-
TAATACGACTCACTATAGGGCCGATATCCGACGGTAGTGTTCGA
GCGTNNNGACGGGCGGTGTGTAC.
18S-rRNA-BC14-
(SEQ ID NO: 69)
TAATACGACTCACTATAGGGCCGATATCCGACGGTAGTGTTGATA
GGTNNNGACGGGCGGTGTGTAC.
18S-rRNA-BC15-
(SEQ ID NO: 70)
TAATACGACTCACTATAGGGCCGATATCCGACGGTAGTGTTGCAT
AGTNNNGACGGGCGGTGTGTAC.
18S-rRNA-BC16-
(SEQ ID NO: 71)
TAATACGACTCACTATAGGGCCGATATCCGACGGTAGTGTTGCG
ATCTNNNGACGGGCGGTGTGTAC.
18S-rRNA-BC17-
(SEQ ID NO: 72)
TAATACGACTCACTATAGGGCCGATATCCGACGGTAGTGTTTCCT
GGTNNNGACGGGCGGTGTGTAC.
18S-rRNA-BC18-
(SEQ ID NO: 73)
TAATACGACTCACTATAGGGCCGATATCCGACGGTAGTGTTACA
GGATNNNGACGGGCGGTGTGTAC.
18S-rRNA-BC19-
(SEQ ID NO: 74)
TAATACGACTCACTATAGGGCCGATATCCGACGGTAGTGTTGTG
GTTGNNNGACGGGCGGTGTGTAC.
18S-rRNA-BC20-
(SEQ ID NO: 75)
TAATACGACTCACTATAGGGCCGATATCCGACGGTAGTGTTTCCA
TTGNNNGACGGGCGGTGTGTAC.
18S-rRNA-BC21-
(SEQ ID NO: 76)
TAATACGACTCACTATAGGGCCGATATCCGACGGTAGTGTTAAC
GCTGNNNGACGGGCGGTGTGTAC.
18S-rRNA-BC22-
TAATACGACTCACTATAGGGCCGATATCCGACGGTAGTGTTTGGT
ATGNNNGACGGGCGGTGTGTAC.
(SEQ ID NO: 77)
18S-rRNA-BC23-
(SEQ ID NO: 78)
TAATACGACTCACTATAGGGCCGATATCCGACGGTAGTGTTGAA
CTGGNNNGACGGGCGGTGTGTAC.
18S-rRNA-BC24-
(SEQ ID NO: 79)
TAATACGACTCACTATAGGGCCGATATCCGACGGTAGTGTTACTT
CGGNNNGACGGGCGGTGTGTAC.
18S-rRNA-BC25-
(SEQ ID NO: 80)
TAATACGACTCACTATAGGGCCGATATCCGACGGTAGTGTTCCA
GTCGNNNGACGGGCGGTGTGTAC.
18S-rRNA-BC26-
(SEQ ID NO: 81)
TAATACGACTCACTATAGGGCCGATATCCGACGGTAGTGTTGTAT
GCGNNNGACGGGCGGTGTGTAC.
18S-rRNA-BC27-
(SEQ ID NO: 82)
TAATACGACTCACTATAGGGCCGATATCCGACGGTAGTGTTCATT
GAGNNNGACGGGCGGTGTGTAC.
18S-rRNA-BC28-
(SEQ ID NO: 83)
TAATACGACTCACTATAGGGCCGATATCCGACGGTAGTGTTGGC
TCAGNNNGACGGGCGGTGTGTAC.
18S-rRNA-BC29-
(SEQ ID NO: 84)
TAATACGACTCACTATAGGGCCGATATCCGACGGTAGTGTTATGC
GAGNNNGACGGGCGGTGTGTAC.
18S-rRNA-BC30-
(SEQ ID NO: 85)
TAATACGACTCACTATAGGGCCGATATCCGACGGTAGTGTTCAG
ATTCNNNGACGGGCGGTGTGTAC.
18S-rRNA-BC31-
(SEQ ID NO: 86)
TAATACGACTCACTATAGGGCCGATATCCGACGGTAGTGTGGTT
GGACNNNGACGGGCGGTGTGTAC.
18S-rRNA-BC32-
(SEQ ID NO: 87)
TAATACGACTCACTATAGGGCCGATATCCGACGGTAGTGTGACA
CTTANNNGACGGGCGGTGTGTAC.
18S-rRNA-BC33-
(SEQ ID NO: 87)
TAATACGACTCACTATAGGGCCGATATCCGACGGTAGTGTGCTAT
GGANNNGACGGGCGGTGTGTAC.
18S-rRNA-BC34-
(SEQ ID NO: 89)
TAATACGACTCACTATAGGGCCGATATCCGACGGTAGTGTGTAA
CCGANNNGACGGGCGGTGTGTAC.
18S-rRNA-BC35-
(SEQ ID NO: 90)
TAATACGACTCACTATAGGGCCGATATCCGACGGTAGTGTGGCA
AGCANNNGACGGGCGGTGTGTAC.
18S-rRNA-BC36-
(SEQ ID NO: 91)
TAATACGACTCACTATAGGGCCGATATCCGACGGTAGTGTGAAC
GACANNNGACGGGCGGTGTGTAC.
RTprimer 2 for 18S rRNA-
(SEQ ID NO: 92)
GATTTGTCTGGTTAATTCCGATAACG.
RTprimer 2 for N gene-
(SEQ ID NO: 93)
CGTGGTCCAGAACAAACCCA.
The RT primer 2 in FIG. 2 refers to
RT primer 2 for N gene.
FP1 for 18SrRNA-
(SEQ ID NO: 94)
CAATAACAGGTCTGTGATGCCCT.
FP2 for 18S rRNA-
(SEQ ID NO: 95)
TGCAATTATTCCCCATGAACGAG.
FP1 for N gene-
(SEQ ID NO: 97)
AGGTGCCATCAAATTGGATGACA.
The FP1 in FIG. 2 refers to FP1 for N gene.
FP2 for N gene-
(SEQ ID NO: 98)
CTGAATAAGCATATTGACGCATAC.
The FP2 in
FIG. 2 refers to FP2 for N gene.
RP-
(SEQ ID NO: 99)
CCGATATCCGACGGTAGTGT.
The RP in FIG. 2
refers to RP for N gene.
IVT-PCR-FP-
(SEQ ID NO: 100)
GTAAAACGACGGCCAGTGAATT.
IVT-PCR-RP-
(SEQ ID NO: 101)
CAGGAAACAGCTATGACCATG.

The following EXAMPLES are provided to illustrate the invention and shall not limit the scope of the invention.

### Example 1

### Party Balloon

In the thirteenth embodiment of the invention, RNA virus particles from human breath were readily precipitated from the interior surface of an inflated party balloon after a one-hour incubation at −20° C. rRNA can be readily detected by RT-PCR in this liquid with no RNA extraction. This collection technique was simple.

### Example 2

### Clinical Trial of the Device

This EXAMPLE shows a test of the efficacy of a device and method of sample collection.

The inventors developed a successful prototype that can copy 18S rRNA from breath with the same efficiency as conventionally sampled RNA. In a clinical study testing this Bubbler™ device, the inventors: (A) applied the Bubbler™ in parallel with conventional nasopharyngeal swab testing for comparison, not for a diagnostic purpose; and (B) characterized the Bubbler™ patient isolate using standard molecular biological diagnostics (Western, PCR, sequencing). The collected sample was evaluated for lung epithelial markers, potential saliva contamination, and viral RNA to determine the most abundant viral regions for improved amplicon design.

The standard of care test for this population is a NP-PCR-based assay, which the patients received besides the investigational test and was used as the gold standard for comparison.

The patients were responsible for paying for anything else besides the Bubbler™ test, this includes the standard of care NP-PCR test performed during their visit and any other charges associated with their visit.

### Example 3

### Patient Population for the Clinical Trial of the Device

A clinical study was conducted at Rhode Island Hospital (RIH) and Miriam Hospital.

Inclusion criteria: The patients recruited for the clinical study were 18 years and older who presented with symptoms consistent with undiagnosed COVID infection. These patients had already undergone a standard of care nasopharyngeal swab and were waiting for their results or would have already received a positive result.

Exclusion criteria: Patients with asthma or COPD exacerbated by COVID infection were excluded from the clinical study because they could not sustain an exhalation into the Bubbler™. Patients who had burns or trauma to the mouth were also excluded. Patients who unable to provide signed consent were excluded.

Study protocol: The patients were first screened for entry into the clinical study. The patients were then shown how to use the Bubbler™ using a different exemplar device during exhalation to a study participant. This demonstration showed the patient what to avoid, such as accidental inhalation. The patients were observed they used the Bubbler™. Upon entry to the clinical study, the patients were offered the opportunity to sign a Specimen Banking form, enabling the storage of what is left of the sample after analysis. Using hospital records, the patient's course was followed by noting demographics, historical and physical exam information, vital signs, laboratory findings, length of stay, mortality, and results of infection-related diagnostics and interventions, and pulse oximeter readings.

The clinical study was blinded as to the results of all testing, especially since samples were stored and run in batches.

Standardized treatment: The clinical study was discussed with the patients who consented at that time. This clinical study was a prospective observational trial. Data from this clinical study was not available to patient providers at the time of provision of care and was not used to influence care or disposition of patients.

Primary outcome: The inventors tested the Bubbler™ device in parallel with conventional nasopharyngeal swab testing. This testing was for comparison, not for a diagnostic purpose.

The inventors characterized the Bubbler™ patient isolates using standard molecular biological diagnostics known to persons having ordinary skill in the biomedical art (Western analysis, PCR, sequencing). The collected sample was evaluated for lung epithelial markers, potential saliva contamination, and viral RNA to determine the most abundant viral regions for improved amplicon design.

Statistical analysis: Some patients tested negative as they were enrolled, which is important for determining the specificity and sensitivity of the assay. The standard NP-PCR-based assay was used as the gold standard comparison in this clinical study. Because even the NP-PCR-based assay has some limitations, a patient's biobanking consent for retaining the exhaled RNA particles was important to ultimately assess results, if a negative NP-PCR-based assay were to be questioned.

This clinical study did not require close follow-up of patients admitted to the hospital with critical illness. Asymptomatic patient may not have yet developed enough COVID-19 viral burden to test positive in either the standard NP-PCR or the Bubbler™ RT-PCR to test positive.

Human subject protection: The clinical study was intended to demonstrate proof-of-concept in the detection of COVID through exhalation and capture of viral particles. The patients' lips only came in contact with clean and unused off the shelf glass pipettes made by Fisher Scientific.

Risks and benefits: There were minimal risks to the patients in this clinical study. The amount of canola oil (90% by volume) and reverse transcriptase reagents (10% v/v) at the bottom of the 15 ml centrifuge tube was 0.6 ml. The dead space volume in the Pasteur pipette within the centrifuge tube was 2.4 ml.

The devices used in the clinical study were made using a sterile construction protocol, are kept sterile until used, and pose little risk of patient contamination. The device was constructed by people wearing gloved hands, sprayed with 70% ethanol and allowed to dry overnight in an ultraviolet (UV) hood. Emulsion is added before use under a sterile protocol.

The likelihood of a participating patient accidentally inhaling the liquid was remote. This risk was further minimized by first demonstrating to the patient the method of using a different exemplar device. The patients' use of the device was observed to ensure safe use during exhalation. The glass Pasteur pipettes being used have historically been used is mouth pipetting in the past. Overall, the risks of the sample collection intervention in this protocol are minimal.

Data safety monitoring: To protect against risks to confidentiality, the information gathering from charts was performed by emergency physicians and trained research coordinators. To ensure data safety, data was entered into an MS-Excel worksheet by patient number. These worksheets were stored on a password-protected computer in the emergency department research office. The clinical staff kept copies of the patients' consents, emergency records, and hospital records in a locked cabinet.

Data collected on each enrolled patient included name, gender, age, medical record number, account identifier, birth date, admission date, pertinent admission conditions, hemodynamics, therapeutic interventions, diagnostics (e.g., culture results, radiography results, blood analysis, pathology summaries), disposition status and hospital date/day. These data could verify the diagnosis of COVID-related infection, to score illness severity, and structure a demographically appropriate control in the event one is needed. Again, all identifying information on each patient was be kept separate from clinical study samples. Data used for analysis did not include patient name, MR number, account identifier, birth date, admission date, or disposition date. Because of adherence to these and other standard confidentiality and data safety protocols, The chance of any breaks in confidentiality were minimal.

### Example 4

TABLE 2
Sequential Organ Failure Assessment Score

| | SOFA Score | | | | |
|---|---|---|---|---|---|
| Variables | 0 | 1 | 2 | 3 | 4 |
| Respiratory | PaO₂/FiO₂: >400 | PaO₂/FiO₂: >400 | PaO₂/FiO₂: >300 | PaO₂/FiO₂: >200 | PaO₂/FiO₂: >100 |
| | SpO₂/FiO₂: >302 | SpO₂/FiO₂: >302 | SpO₂/FiO₂: >221 | SpO₂/FiO₂: >142 | SpO₂/FiO₂: >87 |
| Cardiovascular | MAP ≥ 70 mmHg | MAP ≥ 70 mmHg | Dopamine ≤ 5 or | Dopamine > 5 | Dopamine > 15 |
| (doses in mcg/kg/min) | | | ANY dobutamine | Norepinephrine ≤ 0.1 | Norepinephrine > 0.1 |
| | | | | Phenylephrine ≤ 0.8 | Phenylephrine > 0.8 |
| Liver (bilirubin, mg/dL) | <12 | 12-19 | 2.0-5.9 | 6.0-11.9 | >12 |
| Renal (creatinine, mg/dL) | <12 | 12-19 | 2.0-3.4 | 3.5-4.9 | >5.0 |
| Coaguation | ≥150 | <150 | <100 | <50 | <20 |
| (platelets × 10²/mm³) | | | | | |
| Neurologic | 15 | 13-14 | 10-12 | 5-9 | <5 |
| (GCS score) | | | | | |

According to Sepsis G. a new (or presumed new) increase in SOFA score above baseline in the presence of infection makes the diagnosis of sepsis. Increasing SOFA scores are associated with incremental increase in mortality.
Abbreviations: GCS, Glasgow coma scale; FiO₂, fraction of inspired oxygen; MAP, mean arterial pressure; PaO₂, arterial oxygen pressure; SOFA, sequential organ failure assessment (score); SpO₂, oxygen saturation.

### Example 5

Efficient Amplification of SARS-CoV-2 RNA from Exhaled Breath or Oral Samples without RNA Extraction.

The inventors improved SARS-CoV-2 detection by simplifying the assay and broadening the compartments tested. Then, the inventors designed a clinical study to sample COVID from three points in the respiratory tract. Oral samples by saliva/tongue scrapes or exhaled breath were compared to the traditional nasopharyngeal swab. To simplify the assay, the inventors explored the viability of performing reverse transcription directly on a sample without RNA extraction, eliminating the need to stabilize a sample and allowing the assay to be performed at home. the inventors describe the design and testing of a breathalyzer called the Bubbler™ that directly samples aerosolized particles in exhaled breath.

Results. While SARS-CoV-2 is predominantly sampled in the upper respiratory tract by a nasopharyngeal swab, most fatalities arise from involvement of the lower respiratory tract. As the risk of transmission is a function of viral load in exhaled droplets, there is a strong argument for assaying the viral load in exhaled breath. To assay the SARS-CoV-2 RNA in human breath, the inventors developed a hand-held breathalyzer that reverse-transcribed RNA to DNA at the site of sample collection.

The Bubbler™ was developed as an improved, alternate capture device. The prototype used in the clinical study was a modified 15 ml Falcon tube with a glass straw which allows exhaled breath to be bubbled through an oil/RT mixture emulsion. See FIG. 1(C) and FIG. 6.

Preclinical studies demonstrated that Bubbler™ samples had a similar level of RT-PCR efficiency to RNA extracted from cultured cells. More rRNA could be detected from a single (less than ten seconds) breath than could be detected from conventionally-extracted RNA.

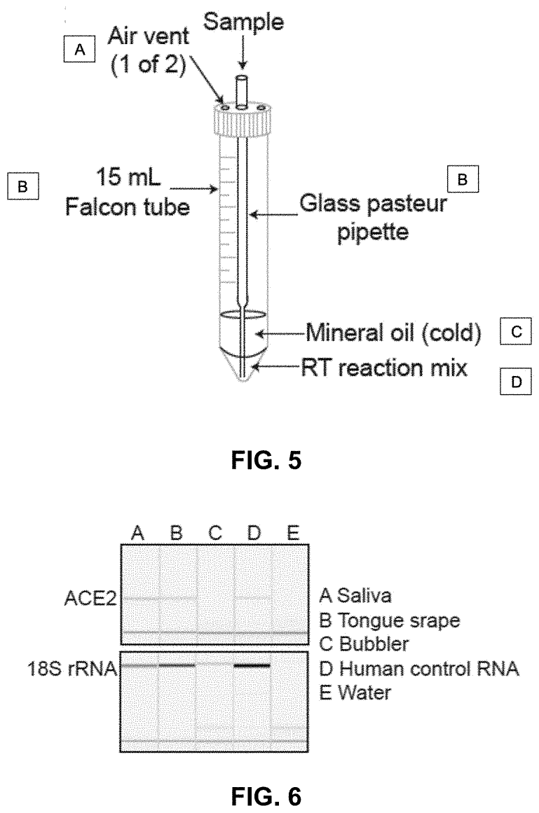

In fourteenth embodiment, the inventors optimized the device and demonstrated that the Bubbler™ could be miniaturized and the RT reaction mixture was stable in the kit for at least two weeks. See FIG. 5.

The inventors tested the Bubbler™ on patients encountered in the emergency department of Rhode Island hospital. The clinical study was intended to explicitly test (a) the diagnostic potential of exhaled breath and (b) the viability of performing the reverse transcription at the site of collection. Performing reverse transcription at the site of collection simplifies the protocol by eliminating the stabilization and RNA extraction steps. Kits were constructed to include one Bubbler™ and two saliva/tongue scrapes as controls. Several experiments were conducted to compare samples collected from the Bubbler™ to the control. Interestingly, samples collected from the tongue scrape were positive for expression of the ACE2 receptor whereas ACE2 signal was undetectable in Bubbler™ samples, suggesting the Bubbler™ and tongue scrape sample RNA from distinct compartments. See FIG. 6.

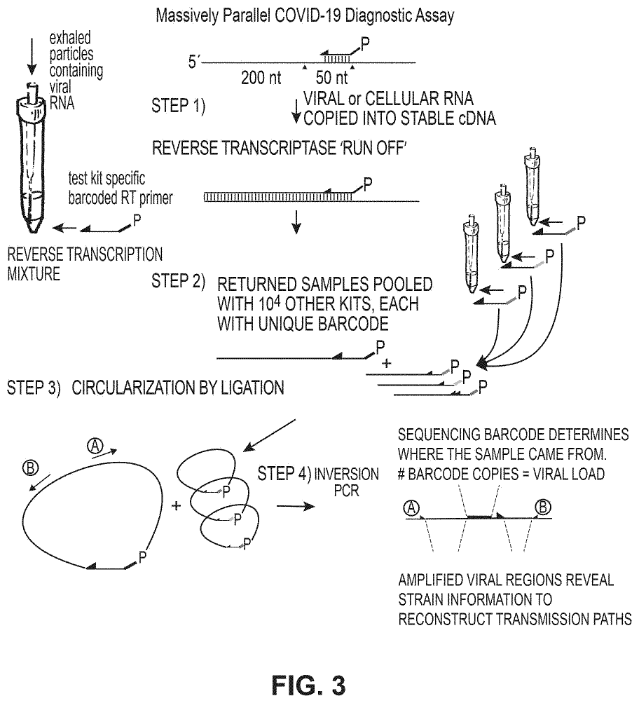

To determine if SARS-CoV-2 could be detected from the Bubbler™, an RT-PCR assay to amplify SARS-CoV-2 RNA was optimized on a commercially available positive control. The optimization yielded RT and PCR primers that performed with similar sensitivity to the CDC primers, N1 and N2. See FIG. 2. Amplification of the housekeeping gene RNase P was used as a sample control. Reverse transcriptase reaction mixtures were added to Bubblers™ and sample tubes and packaged in test kits administered to consenting enrolled patients during their treatment at Rhode Island Hospital. A total of seventy patients were tested over a period of approximately 7 months. See FIG. 3. Each patient was offered enrollment in a study to test the Bubbler™ and tongue scrapes and, as part of the standard emergency department evaluation protocol included a hospital swab PCR test (H-PCR), these results were available for comparison. The positivity rate for all three tests tracked the CDC state wide testing data. See FIG. 3). Both the lab-based tongue scrape PCR (L-PCR) and the Bubbler™-based PCR (B-PCR) returned more positive samples than the H-PCR, presumably due to increased efficiency of the optimized PCR.

Binary classification tests were computed to summarize the comparisons between the three tests deployed in the clinical study. See TABLE 1. The H-PCR test showed a positive predictive value (PPV) of 0.65 compared to the L-PCR test, and the results from H-PCR and L-PCR were significantly different (McNemar's test, p=0.02). The H-PCR showed a PPV of 0.95 for abnormal chest X-rays (positive XR). The H-PCR showed a PPV of 0.69 for confirmed positive Bubbler™ tests. The confirmed positive Bubbler™ tests showed a PPV of 0.94 for positive XRs. Overall, the L-PCR confirmed Bubbler™ results showed equally strong prediction for a positive XR as the H-PCR positive results. However, upon rank-ordering prediction estimates, B-PCR showed stronger prediction for a positive XR finding than the H-PCR results (Z=1.98; p=0.02).

While comparing multiple assays of unknown error rate is limited by a lack of clearly defined true positives, the increased predictive power of the Bubbler™ for COVID-19 cases accompanied by evidence of lower respiratory track involvement, e.g. pneumonia visualized by X-ray, is reminiscent of compartmentalization of influenza. These results position the Bubbler™ as an attractive alternative to bronchoalveolar lavage for sampling the lower respiratory track.

Benchmarking the Bubbler™ against nasopharyngeal swabs and tongue scrapes must consider the possibility that the PCRs performed on these samples are measuring the same amplicon in different contexts, e.g., genome in viral particle; viral transcripts in lysed cells, etc. To better characterize the sample collected by the Bubbler™, the composition of cellular RNAs in exhaled air collected from seventy patients was reanalyzed. RNase P levels were a proxy to compare the ratio of cellular to SARS-CoV-2 RNA in exhaled breath relative to conventionally-collected samples. RNase P is expected to be expressed in every cell, whereas SARS-CoV-2 RNA is presumably localized to airborne viral particles and material released from lysed cells. The data obtained in this EXAMPLE showed the Bubbler™ sample is more weighted towards viral particles as the ratio of CT scores of SARS-CoV-2 to RNase P were over 3-fold higher than observed in the tongue scrape.

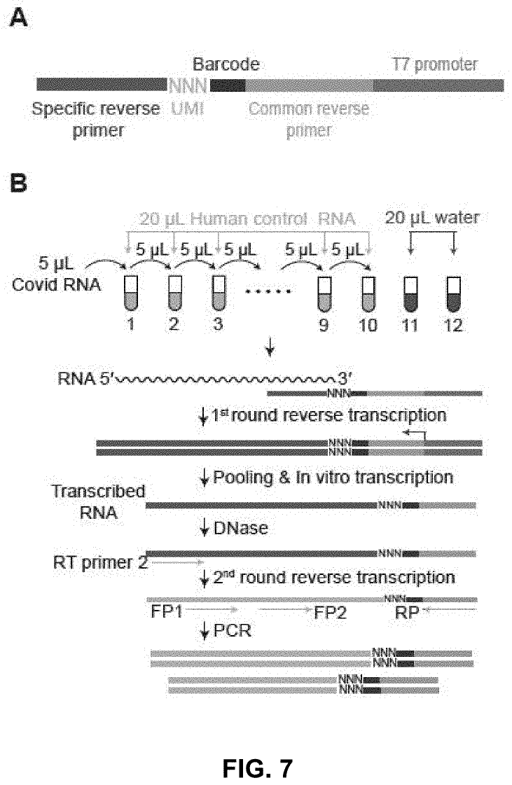

An advantage of performing reverse transcription in the collection tube was to use barcoded cDNA in a high throughput testing scheme. FIG. 7(A). Each RT primer targets a window of RNA but still functions with an additional sequence at the 5′ end. This sequence consisted of a T7 promoter to amplify the signal, a 6-nucleotide sample barcode, and a 3-nucleotide random tag to distinguish unique RT events from duplicates that arise in amplification. To test the detection limit of this assay, barcoded primers were used to test in triplicate a series of ten five-fold dilutions of SARS-CoV-2 and two water-based blanks. Samples are reverse transcribed, pooled and then subjected to a two-step nested PCR strategy. See FIG. 7(B). After sequencing the resulting amplicons, barcodes were counted and associated with individual amplification events. Barcode counts were highly correlated across replicates and with the expected counts. The correlation was lost at the 5th serial dilution corresponding to a detection limit of 334 genomic copies.

Discussion. Through analysis of condensate from a breathalyzer, the inventors conclude that SARS-CoV-2 can be readily detected in human breath. Viral RNA is more enriched in human breath relative to oral samples while content from cells capable of replicating SARS-CoV-2 is present in saliva but absent in human breath. This finding suggests the viral signal detected in the Bubbler™ comes from viral particles. The significance of sampling airborne viral particles is the key advantage to the Bubbler™ over other technologies. Where the Bubbler™ can measure active infections, other techniques cannot distinguish active infections from prior events that have been resolved. An abnormal X-ray can result from damage caused during prior infections, and the CDC's isolation guideline of three months reflected findings of prolonged viral signal in previously infected patients. While patients are no longer infectious, it is difficult to classify these situations as false positives due to the viral fragments present in the cell. However, the inventors found cases of patients with prior infections which tested negative in the Bubbler™ and inconsistently positive in the tongue scrapes.

Besides the Bubbler™ matching the hospital assay in predicting abnormal X-ray results, these results show that the Bubbler™ samples a compartment enriched in SARS-CoV-2 virus which is likely to be a better indicator of current infection than nasopharyngeal swabs.

The United States Centers for Disease Control (CDC) recommends upper respiratory specimens for initial diagnostic testing for SARS-CoV-2 infection. Despite yielding the highest viral loads for the detection of SARS-CoV-2, sample collection by sputum induction is not recommended due to the likelihood of aerosolization. Collection of lower respiratory tract samples from patients with suspected COVID-19 pneumonia is only recommended when an upper respiratory tract sample is negative. See the United States National Institutes of Health Coronavirus Disease 2019 (COVID-19) Treatment Guidelines.

The most common testing for upper respiratory specimens has been the nasopharyngeal swab. However, nasopharyngeal swabs also carry an aerosolization risk as they are so uncomfortable that patients often cough, sneeze or gag during the procedure, e.g., one patient refused conventional swab. Alternative assays such as the Bubbler™ estimate lower respiratory samples, with the safety of an upper respiratory sample. In addition, finding nasopharyngeal swab alternatives can relieve supply chains for the swabs and transport media, reduce the need for personal protective equipment during aerosolization and provide a more comfortable patient experience.

The results of this EXAMPLE show how barcoding can enable high throughput RNA virus testing at a fraction of the cost of conventional testing. Besides the cost-saving and time saving from parallelization, the diagnosis-by-sequencing method enables strain identification, which is useful because more information is learned about transmissibility and possible strain-specific treatment decisions.

### List of Embodiments

Specific compositions and methods of a massively parallel RNA virus diagnostic assay have been presented. The scope of the invention should be defined solely by the claims. A person having ordinary skill in the biomedical art shall interpret all claim terms in the broadest possible manner consistent with the context and the spirit of the disclosure. The detailed description in this specification is illustrative and not restrictive or exhaustive. This invention is not limited to the particular methodology, protocols, and reagents described in this specification and can vary in practice. When the specification or claims recite ordered steps or functions, alternative embodiments might perform their functions in a different order or substantially concurrently. Other equivalents and modifications besides those already described are possible without departing from the inventive concepts described in this specification, as persons having ordinary skill in the biomedical art recognize.

All patents and publications cited throughout this specification are incorporated by reference to disclose and describe the materials and methods used with the technologies described in this specification. The patents and publications are provided solely for their disclosure before the filing date of this specification. All statements about the patents and publications' disclosures and publication dates are from the inventors' information and belief. The inventors make no admission about the correctness of the contents or dates of these documents. Should there be a discrepancy between a date provided in this specification and the actual publication date, then the actual publication date shall control. The inventors may antedate such disclosure because of prior invention or another reason. Should there be a discrepancy between the scientific or technical teaching of a previous patent or publication and this specification, then the teaching of this specification and these claims shall control.

When the specification provides a range of values, each intervening value between the upper and lower limit of that range is within the range of values unless the context dictates otherwise.

### REFERENCES

A person having ordinary skill in the biomedical art can use these scientific references as guidance to predictable results when making and using the invention.

Non-Patent Literature

Duguid, The size and the duration of air-carriage of respiratory droplets and droplet-nuclei. The Journal of Hygiene 44, 471-479 (1946). Early research from different types of exhaled breath (e.g. sneezing, coughing and talking loudly) has demonstrated a wide range of droplet sizes that persist in the air.

Asadi et al., Aerosol emission and superemission during human speech increase with voice loudness. Scientific Reports 9, 2348 (2019). Research from different types of exhaled breath (e.g. sneezing, coughing and talking loudly) has demonstrated a wide range of droplet sizes that persist in the air.

Pasomsub et al., Saliva sample as a non-invasive specimen for the diagnosis of coronavirus disease 2019: a cross-sectional study. Clin. Microbiol. Infect. 27, 285 (2021). Testing strategies for active or prior infection rely on detection of viral RNA or antibodies to the virus. Collection is usually performed in the upper respiratory tract by saliva or nasopharyngeal swab, which have comparable sensitivities (97% agreement).

Wolfel et al., Virological assessment of hospitalized patients with COVID-2019. Nature 581, 465-469 (2020). While samples contain active coronavirus, a recent study suggested influenza was compartmentalized.

Yan et al., Infectious virus in exhaled breath of symptomatic seasonal influenza cases from a college community. Proc. Natl. Acad. Sci. U.S.A 115, 1081-1086 (2018). While samples contain active coronavirus, a recent study suggested influenza was compartmentalized.

Charlson et al., Topographical continuity of bacterial populations in the healthy human respiratory tract. Am. J. Respir. Crit. Care Med. (184), 957-963 (2011). Prior studies failed to detect differences between lung microbiome and the microbiome of the upper respiratory tract.

Hermans &. Bernard, Lung epithelium-specific proteins: characteristics and potential applications as markers. Am. J. Respir. Crit. Care Med. 159, 646-678 (1999). Cellular genes expressed predominantly in the lung include the family of Surfactant-associated proteins (e.g. SP-A). ACE-2 expression is found, but not restricted to the lung.

Li, Wang & Lv, Prolonged SARS-CoV-2 RNA shedding: Not a rare phenomenon. J. Med. Virol. 92, 2286-2287 (2020). An abnormal X-ray can result from damage caused during prior infections, and the CDC's isolation guideline of three months reflected findings of prolonged viral signal in previously-infected patients.

Yu et al., Quantitative detection and viral load analysis of SARS-CoV-2 in infected patients. Clin. Infect. Dis. 71, 793-798 (2020). Despite yielding the highest viral loads for the detection of SARS-CoV-2, sample collection via sputum induction is not recommended due to the likelihood of aerosolization.

Tu et al., Swabs collected by patients or health care workers for SARS-CoV-2 testing. N. Engl. J. Med. 383, 494-496 (2020). Nasopharyngeal swabs carry an aerosolization risk, because they are so uncomfortable that patients often cough, sneeze or gag during the procedure.

Hossain et al., A Massively Parallel COVID-19 Diagnostic Assay for Simultaneous Testing of 19200 Patient Samples.

Textbooks and Technical References

Current Protocols in Immunology (CPI) (2003). John E. Coligan, ADA M Kruisbeek, David H Margulies, Ethan M Shevach, Warren Strobe, (eds.) John Wiley and Sons, Inc. (ISBN 0471142735, 9780471142737).

Current Protocols in Molecular Biology (CPMB), (2014). Frederick M. Ausubel (ed.), John Wiley and Sons (ISBN 047150338X, 9780471503385).

Current Protocols in Protein Science (CPPS), (2005). John E. Coligan (ed.), John Wiley and Sons, Inc.

Immunology (2006). Werner Luttmann, published by Elsevier.

Janeway's Immunobiology, (2014). Kenneth Murphy, Allan Mowat, Casey Weaver (eds.), Taylor & Francis Limited, (ISBN 0815345305, 9780815345305).

Laboratory Methods in Enzymology: DNA, (2013). Jon Lorsch (ed.) Elsevier (ISBN 0124199542).

Lewin's Genes XI, (2014). published by Jones & Bartlett Publishers (ISBN-1449659055).

Molecular Biology and Biotechnology: a Comprehensive Desk Reference, (1995). Robert A. Meyers (ed.), published by VCH Publishers, Inc. (ISBN 1-56081-569-8).

Molecular Cloning: A Laboratory Manual, 4th ed., Michael Richard Green and Joseph Sambrook, (2012). Cold Spring Harbor Laboratory Press, Cold Spring Harbor, N.Y., USA (ISBN 1936113414).

The Encyclopedia of Molecular Cell Biology and Molecular Medicine, Robert S. Porter et al. (eds.), published by Blackwell Science Ltd., 1999-2012 (ISBN 9783527600908).

The Merck Manual of Diagnosis and Therapy, 19^{th} edition (Merck Sharp & Dohme Corp., 2018).

Pharmaceutical Sciences 23^{rd} edition (Elsevier, 2020).

1. A device, comprising:
(1) a tube at the top, into which a subject can blow a breath; and
(2) a receptacle at the bottom of the device;
wherein the tube and the receptacle are fitted together;
wherein the receptacle contains an oil/water mixture, wherein the oil does not inhibit a reverse transcriptase reaction;
wherein the oil/water comprises reverse transcriptase, primers for a reverse transcriptase reaction, and reagents and buffers for a reverse transcriptase reaction.

2. The device of claim 1, wherein the oil in the oil/water mix is canola oil or mineral oil.

3. The device of claim 1, wherein the reverse transcription in the receptacle converted the RNA in the sample to stable, molecularly barcoded cDNA.

4. The device of claim 3, wherein the stable, molecularly barcoded cDNA is compatible with downstream large-scale sequencing-based parallel diagnostics.

5. The device of claim 1, for use for use as a screen for RNA virus in human breath.

6. The device of claim 1, for use as a screen for DNA virus in human breath.

7. The device of claim 1, for use as a screen for respiratory virus in human breath.

8. The device of claim 7, wherein the respiratory virus is COVID-19 (SARs-CoV-2).

9. The device of claim 1, for use as a screen for airborne virus in the environment, wherein a vacuum pump is applied an air vent.

10. The device of claim 1, for use in detecting nonviral nucleic acid in exhaled breath.

11. The device of claim 1, for use in detecting aerosolized DNA in the environment.

12. The device of claim 1, for use in collecting a sample that is used for sequencing to identify strain of virus.

13. A device, comprising:
(1) a tube at the top, into which a subject can blow a breath; and
(2) a receptacle at the bottom of the device;
wherein the receptacle is a balloon from which RNA virus can be collected.

14. A method for reverse transcribing RNA from airborne SARs-CoV-2 viral particles into a sample-specific barcoded cDNA, comprising the steps of:
(1) obtaining deep-breath sample from a suspected COVID-19 patient;
(2) reverse transcribing the viral RNA to viral cDNA using sample-specific barcoded primers.

15. The method of claim 14, further comprising the step of:
(3) pooling the samples for analysis by a massively parallel assay.
